(19) United States
(12) Patent Application Publication
(10) Pub. No.: US 2006/0105321 A1
(43) Pub. Date: May 18, 2006

(54) Methods for evaluating cell membrane properties

(75) Inventors: Alan B. Moy, Iowa City, IA (US); James E. Bodmer, Iowa City, IA (US); Er-Wei Bai, Iowa City, IA (US); Anthony English, Knoxville, TN (US)

Correspondence Address: FULBRIGHT & JAWORSKI L.L.P., 600 CONGRESS AVE., SUITE 2400, AUSTIN, TX, 78701, US

(73) Assignee: The University of Iowa Research Foundation

(21) Appl. No.: 11/192,322

(22) Filed: Jul. 28, 2005

Related U.S. Application Data
(60) Provisional application No. 60/591,762, filed on Jul. 28, 2004.

(51) Int. Cl. C12Q 1/00; G06F 19/00

(52) U.S. Cl. 435/4; 702/19

(57) ABSTRACT

Methods for measuring cell membrane property are disclosed. A computer readable medium includes instructions for deriving from transcellular impedance a measurement of a cell membrane property and provides the stability and error of the measurement. The derivation from the transcellular impedance includes the real and imaginary components of the transcellular impedance and also uses a geometric shape to model the shape of the cell. Alternatively, the derivation from the transcellular impedance includes magnitude and complex components. The measurements include the membrane capacitance, membrane resistance, and/or cell adhesion.

This application claims priority to, and incorporates by reference, U.S. Provisional Patent Application Ser. No. 60/591,762 entitled “EVALUATING CELL MEMBRANE PROPERTIES,” which was filed on Jul. 28, 2004.

### STATEMENT AS TO RIGHTS TO INVENTIONS MADE UNDER FEDERALLY-SPONSORED RESEARCH AND DEVELOPMENT

Aspects of this invention were made with government support of the National Science Foundation, grant number BES-0238905; the American Heart Association, grant numbers 0265029B and 0256019Z; and the National Institute of Health, grant number GM61732. Accordingly, the government may have certain rights in this invention.

### BACKGROUND OF THE INVENTION

1. Field of the Invention

The present invention relates to cell membrane properties. More specifically, the present invention determines cell membrane properties from measurements such as transcellular impedance measurements.

2. Description of Related Art

Transendothelial cell monolayer impedance measurements can be a complicated and sensitive function of cell-cell adhesion, cell-matrix adhesion, and membrane capacitance. Endothelial cell monolayer impedance measurements have the potential to provide valuable insight into a number of cellular processes. The adhesion of endothelial cells to each other and an underlying matrix, for example, can be critical to the optimal development of tissue engineered vascular constructs and the etiology of many cardiovascular, pulmonary and renal diseases. The ability to quantify changes in measured electrical impedance resulting from changes in endothelial cell-cell impedance, cell-matrix impedance, and membrane capacitance, therefore, has a number of important applications in medicine and biotechnology.

There have been several different techniques to measure barrier function and cell adhesion in the art. For example, permeability has been used to measure barrier function in which the diffusion of a macromolecule is monitored across a cultured monolayer grown on a filtered-support. However, permeability represents an indirect measurement of cell-cell and cell-matrix adhesion. Further, there is a time lag between loss of cell adhesion and diffusion of a macromolecule. Another technique used to measure epithelial barrier function is a voltage clamp system, which uses direct current. However, this technique lacks the sensitivity to measure the low resistance of cultured endothelial cells and cannot generate numerical models to resolve cell-cell and/or cell-matrix adhesion.

Other conventional methods to evaluate cell adhesion use immunocytochemistry to measure focal contacts. However, there are several shortcomings with this approach. Mainly, the studies are performed on fixed cells. As such, the method reflects an anatomical characteristic rather than a physical parameter of adhesion. Another method uses quantitative interference reflection microscopy, (RM), to quantify cell-matrix adhesion in living cells. However, cell-matrix adhesion represents only a fraction of cytoskeletal-membrane properties of cultured cells, and therefore, the IRM technique does not accurately characterize the cells. Other techniques utilize fluorescent or radiolabeled reagents to measure cell attachment. However these techniques do not detect constitutive changes in cell adhesion in response to physiological or pharmacological stimuli once cells have established their attachment.

Additionally, electrical measurements have been utilized in the art to dynamically and quantitatively measure cell adhesion in cultured cells. One example is a technique called electrical cell substrate impedance sensor (ECIS) that measures transcellular impedance across confluent cultured monolayers grown on a microscopic electrode in which the diameter of the electrode exceeds the dimension of a single cell. However, the limitation of this measurement is that it does not provide a direct experimental measurement of cell-cell and cell-matrix adhesion and cell membrane capacitance because of the size of the sensor. These spatial cell membrane properties may have to be indirectly derived by numerical modeling the measured transcellular impedance. Further, other techniques have tried to derive direct measurements of cell-matrix adhesion, membrane capacitance and membrane resistance by measuring transcellular impedance across cell-covered electrodes in which the diameter of the electrode is below the dimension of a single cell. These experimental measurements are acquired in non-confluent cultured monolayers, and there is no control how the cell is positioned across the electrode surface. With these techniques, measurement of cell-cell adhesion cannot be determined.

Also, the physiological behavior of cultured cells under non-confluent conditions is quite different from the behavior in confluent cultured monolayers. In order to derive spatial measurements of cell-cell and cell-matrix adhesion and membrane capacitance from the ECIS system, prior techniques have introduced a numerical model that derives these membrane parameters by identifying the optimal solutions that account for the experimental data. While the numerical model was applied for epithelial cells, endothelial cells, and cultured fibroblasts, the technique does not provide for stability, accuracy, and precision of the model solutions, which is critical to the end user. Generally, the current modeling techniques are based on fixed assumptions that the cell shape should be modeled as a disk shape, even though this shape did not approximate the native shape of cultured cells. Also, the prior technique does not provide an analysis of error or bias that would provide a confidence assessment of the model solutions. Further, the modeling techniques relied on the real value of the impedance or resistance, and ignored the imaginary value, which provides an approach to evaluate model stability, accuracy, and precision.

Any shortcoming mentioned above is not intended to be exhaustive, but rather is among many that tends to impair the effectiveness of previously known techniques for characterizing cell membranes; however, shortcomings mentioned here are sufficient to demonstrate that the methodologies appearing in the art have not been satisfactory and that a significant need exists for the techniques described and claimed in this disclosure

### SUMMARY OF THE INVENTION

Shortcomings of the prior art are reduced or eliminated by techniques disclosed here. These techniques are applicable to a vast number of applications including many applications requiring measurements of transcellular impedance across cultured cells grown on a microsensor to derive measurements of cell-cell and cell-matrix adhesion and membrane capacitance. For instance, the techniques may be applied to any biomedical field in which it is important to measure cell adhesion and motility such as cardiology, immunology, cancer research, tissue engineering, toxicology, drug discovery, or other pharmaceutical fields of research.

In one respect, the invention is a computer readable medium. Computer executable instructions from the computer readable medium provides for determining a cell membrane property, such as cell adhesion, from a transcellular impedance measurement. The cell membrane property includes cell membrane capacitance and cell membrane resistance. Further, the instructions provide for determining a stability and precision of the measurement. Additionally, a statistical precision of the measurement can be determined.

In other respects, the invention is a computer readable medium including computer-executable instructions. The instructions provide for determining a cell membrane property from a measurement utilizing real and imaginary data pertaining to transcellular impedance and using a geometric shape other than a disk to model the shape of a cell.

In another respect, the invention is a computer readable medium including computer-executable instructions. The instructions provide for determining membrane capacitance and a measurement of membrane resistance from a transcellular impedance measurement.

In yet another respect, the invention includes a method for determining from transcellular impedance a measurement of a cell membrane property. The cell membrane property may include cell adhesion. In other respects, the cell membrane includes cell membrane resistance or cell membrane capacitance. The method also includes determining a confidence assessment of the measurement. The confidence assessment includes determining a stability, a precision, or a statistical precision of the measurement.

As is known in the art, computer readable medium may be associated with a computer, a computer file, a software package, a hard drive, a floppy, a CD-ROM, a hole-punched card, an instrument, an Application Specific Integrated Circuit (ASIC), firmware, a “plug-in” for other software, web-based applications, RAM, ROM, or any other type of computer readable medium. This list is not by way of limitation.

Other features and associated advantages will become apparent with reference to the following detailed description of specific embodiments in connection with the accompanying drawings.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a block diagram of a cellular impedance spectrogram data collection system in accordance with embodiments of this disclosure.

FIG. 2 is a profile view of a cultured cell media, in accordance with embodiments of this disclosure.

FIG. 3 is a flowchart showing steps of a method, in accordance with embodiments of this disclosure.

FIG. 4 is a block diagram showing conductance paths of the endothelial cells, in accordance with embodiments of this disclosure.

FIG. 5 is a circuit diagram for a cellular impedance, in accordance with embodiments of this disclosure.

FIG. 6 shows a real and imaginary voltage component of a naked electrode and a cell covered electrode, in accordance with embodiments of this disclosure.

FIG. 7 shows a voltage covariance matrix, in accordance with embodiments of this disclosure.

FIG. 8 shows errors associated with a constant current, in accordance with embodiments of this disclosure.

FIG. 9 shows a fit to a resistance and reactance using error estimates, in accordance with embodiments of this disclosure.

FIG. 10 shows the results of a simulation, in accordance with embodiments of this disclosure.

FIG. 11 shows the results of a simulation, in accordance with embodiments of this disclosure.

FIG. 12 shows results from analysis of a cell covered electrode, in accordance with embodiments of this disclosure.

FIG. 13 shows the results of a simulation, in accordance with embodiments of this disclosure.

FIG. 14 shows a model of current flow around a cell, in accordance with embodiments of this disclosure.

FIGS. 15A-15D show curve fitting of calculated resistance with measured resistance expressed as a ratio of a cell-covered to a cell-free electrode, in accordance with embodiments of this disclosure.

FIG. 16 shows an EMAS front panel depicting the remaining Zₑᵣᵣₒᵣ after a parameter fit between a calculated and an experimental data, in accordance with embodiments of this disclosure.

FIG. 17 shows estimations of real data solution parameters and Chi² values based on modeling a data subset between 25 Hz and 60 kHz, in accordance with embodiments of this disclosure.

FIG. 18 shows correlation between numerical solution parameters and reduced Chi² for imaginary data, in accordance with embodiments of this disclosure.

FIG. 19 shows estimations of magnitude data solution parameters and Chi² values based on modeling a data subset between 25 Hz and 60 kHz, in accordance with embodiments of this disclosure.

FIG. 20 shows estimations of complex data solution parameters and Chi² values based on modeling a data subset between 25 Hz and 60 kHz, in accordance with embodiments of this disclosure.

FIG. 21 shows a graph that compares the mean (±SE) solution parameters based on real, magnitude, imaginary, and complex data of the cell covered and the naked electrode, in accordance with embodiments of this disclosure.

FIG. 22 shows a graph that compares the mean (±SE) solution parameters based on real, magnitude, imaginary, and complex data of the cell covered and the naked electrode, in accordance with embodiments of this disclosure.

FIG. 23 shows a solution parameter estimates and Chi² when the value of cell membrane resistance, Rₘ (Ω/cm²), is incorporated within the numerical model and fixed at various values, in accordance with embodiments of this disclosure.

### DESCRIPTION OF ILLUSTRATIVE EMBODIMENTS

Endothelial cell-cell and cell-matrix adhesion may be used, for example, in regulating endothelial barrier function, angiogenesis, atherosclerosis, and metastatic cancer. Measurement of transendothelial impedance across cultured endothelial monolayers inoculated on a microscopic electrode has been increasingly utilized as a popular technique to measure endothelial integrity. Yet, since the electrode is larger than the diameter of a single cell, the electrical current conducts through three pathways that are impeded by cell-cell adhesion, cell-matrix adhesion, and membrane capacitance. Breakdown of transendothelial impedance into the cell-membrane parameters cannot be directly measured but, instead, may be derived by mathematical modeling. In one embodiment, accurate and precise measurements of cell-membrane properties may be dependent on numerical solution parameters derived by optimization procedures that exhibit minimal numerical error and stability. The present disclosure quantified and uses several strategies to assess modeling stability and error based on a LM-NLS optimization algorithm of the real and imaginary components of transendothelial impedance. LM-NLS optimization algorithms are considered a robust and well-recognized optimization approach to execute numerical analyses of nonlinear computational problems.

In other embodiments, the present disclosure provides for using alternating currents to measure endothelial barrier function by growing cultured cell on a microelectrode, which offers several advantages. First, the alternating current approach may resolve endothelial barrier function in a dynamic and quantitative fashion. Secondly, the alternating current technique described in the present disclosure may generate data used by mathematical models that derive the contribution of cell-cell and cell-matrix adhesion on barrier function and may also predict cell behavior. Mathematical modeling of transcellular impedance into measurements of cell-cell and cell-matrix adhesion allows for evaluation of unique adhesion receptors at cell-cell and cell-matrix sites. This is important for distinguishing the signal transduction pathway that is targeted. Particularly, the mathematical modeling can be done simultaneously to evaluate the changes in cell-cell and cell-matrix adhesion to understand each adhesion sites contribution within the context of experimental changes in basic cell functions. Also, due to receptor-ligand initiation of signal transduction pathways in cultured endothelial cells occurring rapidly as well as the ambiguity of whether spatial changes in cell adhesion occur from primary targets from signal or from secondary effects, the mathematical model may also allow for the primary and secondary effects to be resolved. Further, mathematical modeling may reduce the time and expense of research.

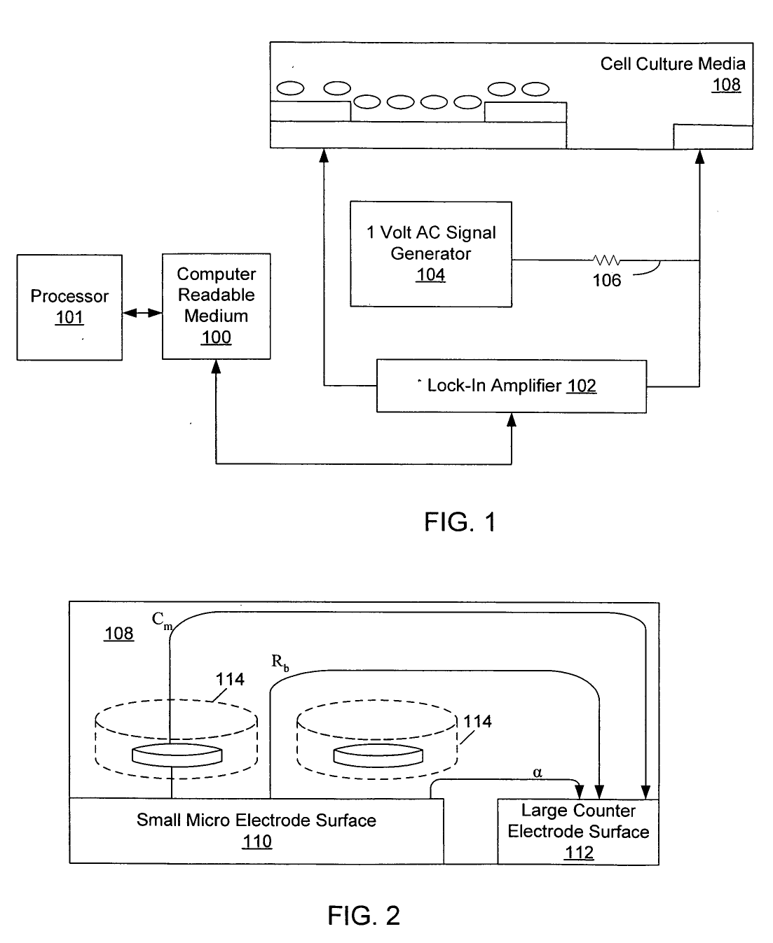

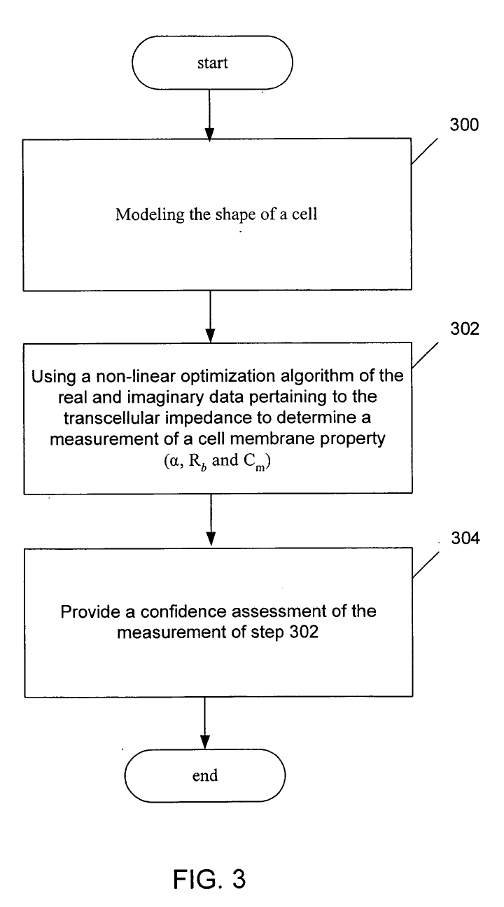

In one embodiment, a computer readable medium (e.g. a software environment) may include instructions for simulating and analyzing cell-covered monolayer based on collected impedance spectroscopic data to derive solutions such as Cₘ, R_{b}, and alpha (α), where Cₘ represents the impedance from transcellular electrical conductance, R_{b} represents the impedance due to cell-cell adhesion, and α represents the impedance due to cell-matrix adhesion. The data collection technique may be implemented on the computer readable medium 100, as shown in FIG. 1. In one embodiment, computer readable medium 100 may be embodied internally or externally on a hard drive, ASIC, CD drive, DVD drive, tape drive, floppy drive, network drive, flash, or the like. Computer readable medium 100 may be coupled to processor 101 configured to execute instructions from computer readable medium 100. For example, processor 101 may implement the steps shown in FIG. 3. In one embodiment, processor 101 is a personal computer (e.g., a typical desktop or laptop computer operated by a user). In another embodiment, processor 101 may be a personal digital assistant (PDA) or other handheld computing device.

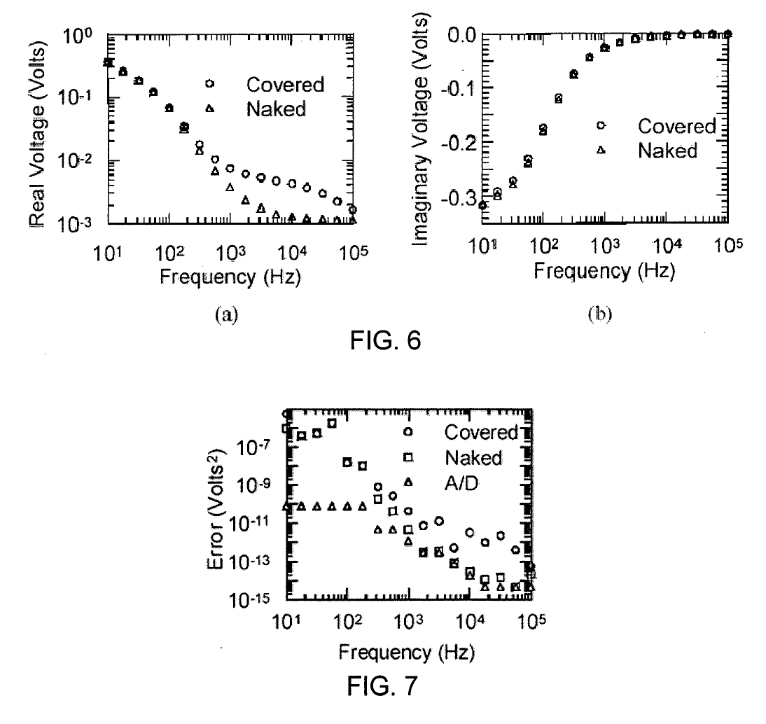

In some embodiments, processor 101 may be a networked device and may constitute a terminal device running software from a remote server, wired or wirelessly. Input from a user or other system components, may be gathered through one or more known techniques such as a keyboard and/or mouse. Output, if necessary, may be achieved through one or more known techniques such as an output file, printer, facsimile, e-mail, web-posting, or the like. Storage may be achieved internally and/or externally and may include, for example, a hard drive, CD drive, DVD drive, tape drive, floppy drive, network drive, flash, or the like. Processor 101 may use any type of monitor or screen known in the art, for displaying information, such as but not limited to, figures similar to FIGS. 6-23. For example, a cathode ray tube (CRT) or liquid crystal display (LCD) can be used. One or more display panels may also constitute a display. In other embodiments, a traditional display may not be required, and processor 410 may operate through appropriate voice and/or key commands.

FIG. 1 also includes a cell culture media 108, such as an electrolyte solution coupled to computer readable medium 100. Processor 101 may provide instructions from computer readable medium 100 to lock-in amplifier 102 for measuring the resultant in-phase and out-of-phase current after a current reference is applied (via AC signal generator 104). The resistor 106, with a value of approximately 1 MΩ may be added in series to approximate a constant current source from one measurement to the next.

Particularly, computer readable medium 100 may receive an input of a previously collected impedance spectrogram of a naked electrode (received prior to or after an experiment is implemented). As describe herein, a naked electrode is an electrode that does not contain a cellular monolayer. The impedance spectrogram of the naked electrode may be used as a model calibration. In other embodiments, computer readable medium 100 may also receive impedance spectrogram of cell-covered electrodes. The cell-covered electrode response may be used in a mathematical comparison with a simulated cell covered response in which a curve-fitting algorithm may find the optimum set of parameters that minimize the error between the cell-covered electrode response and the simulated cell-covered response using a curve fitting algorithm, such as, but not limited to, the Levenberg-Marquardt Non-Linear Least Squares algorithm. Next, computer readable medium 100 may provide instructions to measure the cell membrane property. For example, the instructions may include measuring cell adhesion (e.g. cell-cell adhesion and/or cell-matrix adhesion), cell membrane capacitance, and/or cell membrane resistance. Referring to FIG. 2, cultured cells 114 within cell culture media 108 are modeled as thin disks. It is noted that other geometries may be used to model the cultured cells. In a non-limiting example, the cells may be modeled as squares, rectangles, parallelograms, triangles, ellipsoids, and the likes. Cells 114 may be hovering some distance above the attachment substratum 110 and 112 and may be attached to each other by forming a monolayer with a certain cell-cell attachment distance. As cultured cells 114 change shape due to exogenously applied challenges, the impedance may be affected at various frequencies. Further, current flowing below the monolayer may be affected by the monolayer attachment height and is characterized by the parameter alpha, (α), as shown in FIG. 2. The impedance due to flow between the cells, representative of the endothelial barrier function is labeled R_{b} and the impedance due to capacitive or imaginary component current flow across the cellular monolayer is labeled Cₘ.

Computer readable medium 100 may also provide a confidence assessment of the measurement. A non-limiting example includes graphs depicting the raw data for both the naked and cell-covered responses, which may be provided in several formats. Graphs of the simulated cell covered response as well as representations of error between the experimental data and the simulated response may also be provided. Further, the optimum fitting parameters as a result of the assessment may be provided along with a Chi² value giving a qualitative measure of error. In some embodiments, the confidence assessment may include an stability assessment. Stability, as used in this disclosure, determines is the measurement is reproducible and steady over a period time and frequency. In other embodiments, the confidence assessment may include a measurement precision assessment. Measurement precision, as used in this disclosure, determines how much resolution the measurement is made with. Alternatively, the confidence assessment may include a statistical precision. Statistical precision, as used in this disclosure, defines the quality of the measurement being reproducible.

A flowchart showing steps for simulating and analyzing a cell-covered monolayer according to embodiments of the invention is shown in FIG. 3. In step 300, a cell is first modeled in a geometric shape, such as a disk. The cell may be a cultured cell grown on a microsensor (e.g., a microscopic biosensor, a microelectrode, etc.). In some embodiments, using a non-linear optimization algorithm, such as a Levenberg-Marquardt non-linear optimization algorithm or a least-square non-linear optimization algorithm, and the real and imaginary data pertaining to the transcellular impedance, a measurement of the cell membrane property may be obtained, as shown in step 302. In particular, the cell membrane property may include, but is not limited to cell-cell adhesion, cell-matrix adhesion, cell membrane capacitance, and cell membrane resistance.

The measurement done in step 302 may be assessed, as shown in step 304. In one embodiment, a confidence assessment of the measurement may include, but is not limited to an stability assessment, a precision assessment, and a statistical precision assessment.

### EXAMPLES

The following examples are included to demonstrate specific embodiments of this disclosure. It should be appreciated by those of skill in the art that the techniques disclosed in the example that follow represent techniques discovered by the inventors to function well in the practice of the invention, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the invention.

### Example 1

### Endothelial Cells—Electric Cell-Substrate Impedance Sensor Parameter

Electrical impedance measurements coupled with biotransport and cellular may be useful tools for investigating endothelial cell barrier function in terms of receptor-ligand interactions and signal transduction pathways. A relatively recently developed cellular impedance sensor, referred to as the Electric Cell-Substrate Impedance Sensor or ECIS, has been used to study endothelial cell physiology. Using this sensor, endothelial cell barrier function has been examined using fixed frequency impedance measurements. In addition, this method has been applied to several studies of cellular micromotion in tissue culture.

By making impedance measurements over a range of frequencies, it is possible to numerically evaluate more than one barrier function parameter estimate. The importance of considering both paracellular and subcellular transport paths in the interpretation of transepithelial electrical resistance (TER) measurements has been underscored by some in the field of art. Using a circular cell model geometry, prior art techniques have estimated both paracellular and transcellular pathways from ECIS impedance measurements made over a range of frequencies. Further, conventional methods have used similar impedance measurements to examine the contribution of actin-myosin contraction to the modulation of endothelial cell focal adhesion caused by histamine and thrombin.

The following example examines the sources of random and deterministic noise that contributes to a set of cellular barrier function parameter error bounds based on the ECIS method. In addition, the cell membrane capacitance is considered as an additional parameter to be determined experimentally. The source of electrical impedance fluctuations have been examined experimentally using porcine pulmonary artery endothelial cells (PPAC) and numerically using a multi-response Levenberg-Marquardt non-linear optimization algorithm and Monte Carlo simulations.

1. Materials and Methods

Pulmonary endothelial cells were isolated from porcine pulmonary arteries obtained from a local abattoir. After resecting a couple of inches of a pulmonary artery, each end was clamped using a hemostat and the artery was quickly dipped in 70% ethanol and then rinsed thoroughly with M199 (GibcoBRL, Gaithersburg, Md.). The arteries were then transferred back to the lab in M199 containing penicillin (approximately 100 U/mL) and streptomycin (approximately 100 μg/mL) (GibcoBRL, Gaithersburg, Md.). Each artery was longitudinally dissected open with sterile scissors. The intimal layer of endothelial cells were carefully scrapped from the luminal surface using a sterile scalpel and transferred to a 35 mm petri dish by gently tapping the scalpel blade on the petri dish surface. The 35 mm tissue culture dish contained approximately 2 mL of 50:50 conditioned M199. All cells were cultured using M199 containing 1% penicillin and streptomycin, supplemented with 1% L-glutamine (GibcoBRL, Gaithersburg, Md.), 1% BME amino acids (Sigma, St. Louis, Mo.), 2% BME vitamins (Sigma, St. Louis, Mo.) and 10% fetal bovine serum (Hyclone, Logan, Utah). After about 4 hours, the media from the 35 mm culture dishes was removed and replaced with 2 ml of fresh conditioned media. The culture was maintained until the endothelial cells neared confluence after approximately 7 to 10 days. The endothelial cells were then transferred into a 60 mm tissue culture dish when near confluence. Approximately 1 week later, when the endothelial cells in the 60 mm dish had reached confluence, each dish was transferred into one 100 mm tissue culture dish. The endothelial cells were then transferred once a week at a ratio of 1:3 or 1:4. Cell lines were not transferred beyond the 12^{th} transfer. Porcine pulmonary artery endothelial cells between transfers four and eight were used for this example. Cultures were identified as endothelial cells by their characteristic uniform morphology, uptake of acetylated LDL, and by indirect immunofluorescent staining for Factor VIII.

A one mg/mL fibronectin (BD Biosciences, San Diego, Calif.) solution was prepared by dissolving 1 mg of fibronectin in 1 mL of distilled deionized water. Five well gold electrodes (Applied Biophysics, Troy, N.Y.), were coated with fibronectin using a 100 μg/ml solution that was prepared by thawing a frozen 40 μl aliquot of the 1 mg/mL fibronectin stock and adding 360 μl of sterilized PBS containing Ca⁺⁺ and Mg⁺⁺. Endothelial cells grown to confluence in a 100 mm culture dish were trypsinized using 0.05% trypsin (GibcoBRL) and counted using a hemocytometer. Counts of 30, 34, 41, and 35 were obtained in each of four quadrants, yielding an average of 3.5 million cells in the entire 100 mm dish. The cells were then spun down, the trypsin drawn off, and then re-suspended in 10 ml M199. From this cell solution, 400 μl were added to the electrode wells maintaining a seeding density of approximately 10⁵ cells/cm². Endothelial cells were permitted to attach over a 16 hour period in an incubator. Pictures of each well were taken before and after cell seeding. The entire surface of each well was carefully examined for endothelial cell confluence and cobblestone morphology.

2. Electrical Impedance Model and Measurement

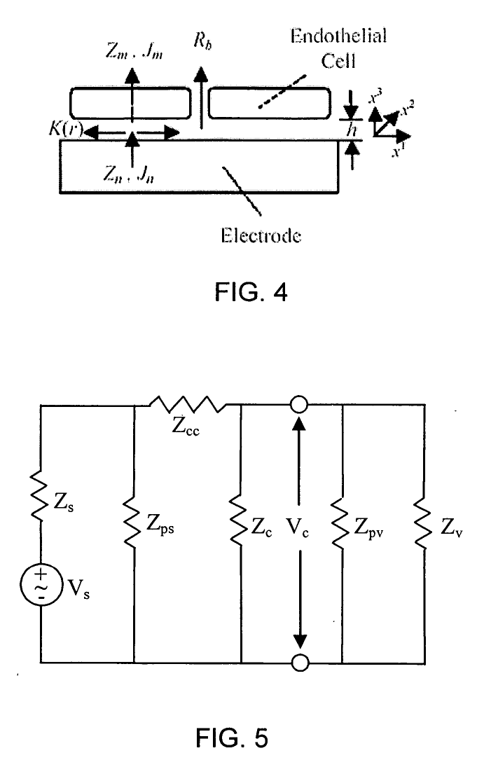

FIG. 4 shows a schematic illustration of the endothelial cell conductance paths. The impedance Rb represents the intercellular or paracellular resistance path between adjacent cells. As adjacent cells pull apart, or the degree of cell-cell adhesion decreases, Rb decreases. The transcellular pathway consists of two membranes in series, each with a capacitance Cm, giving impedance Zm. An additional resistance that represents the resistive path underneath the cell depends on the media solution resistivity (p) and the subcellular separation distance (h). Increasing cell-matrix adhesion that decreases the subcellular space increases the subcellular resistance.

To determine the potential field equation underneath a cell, a subcellular current density, K, and potential, V, which varies only in the x¹/x² plane was considered, as shown in FIG. 4. Between the electrode and the basal surface of the cell the current and potential are assumed constant in the x³ direction. The current densities at the electrode electrolyte interface and the subcellular interface, Jₙ and Jₘ respectively, are directed in the x³ direction. The governing field equations are

$$\begin{matrix}E ( {x}^{1} , {x}^{2} ) = \frac{ρ}{h} K ( {x}^{1} , {x}^{2} ) ⇒ - {∇}_{{x}^{1} {x}^{2}} V ( {x}^{1} , {x}^{2} ) = \frac{ρ}{h} K ( {x}^{1} , {x}^{2} ) , & ( 1 ) \\ {V}_{n} - V ( {x}^{1} , {x}^{2} ) = {Z}_{n} {J}_{n} ( {x}^{1} , {x}^{2} ) , & ( 2 ) \\ V - {V}_{m} ( {x}^{1} , {x}^{2} ) = {Z}_{m} {J}_{m} ( {x}^{1} , {x}^{2} ) , & ( 3 ) \\ {V}_{{x}^{1} {x}^{2}} \cdot J ( {x}^{1} , {x}^{2} ) = {J}_{n} ( {x}^{1} , {x}^{2} ) - {J}_{m} ( {x}^{1} , {x}^{2} ) , and & ( 4 )\end{matrix}$$

where p is the solution resistivity (Ω-m), Zₙ(v) the specific impedance of the electrode-electrolyte interface (Q-m²), and Zₙ(v) is the specific impedance of the cell membrane (Ω-m²). These equations are equivalent to the partial differential equation:

$$\begin{matrix}- {∇}^{2} V - {γ}^{2} V + β = 0 , \\ where & ( 5 ) \\ {γ}^{2} = \frac{ρ}{h} ( \frac{1}{{Z}_{n}} + \frac{1}{{Z}_{m}} ) and β = \frac{ρ}{h} ( \frac{{V}_{n}}{{Z}_{n}} + \frac{{V}_{m}}{{Z}_{m}} ) . & ( 6 )\end{matrix}$$

By redefining the scalar potential function with an additive constant

$$V = V - \frac{β}{{γ}^{2}} \qquad ( 7 )$$

Eq. 5 becomes the Helmholtz equation
∇²V′+γ²V′=0 (8)
In cylindrical coordinates x¹=r, the Laplacian operator takes the form

$${∇}^{2} = \frac{\partial }{\partial r} \frac{1}{r} \frac{\partial }{\partial r} . \qquad ( 9 )$$

The closed form solution is as known in the art, for example,

$$\frac{1}{{Z}_{c}} = \frac{1}{{Z}_{n}} [ \frac{{Z}_{n}}{{Z}_{n} + {Z}_{m}} + \frac{\frac{{Z}_{m}}{{Z}_{n} + {Z}_{m}}}{\frac{γ {r}_{c}}{2} \frac{{I}_{0} ( γ {r}_{c} )}{{I}_{1} ( γ {r}_{c} )} + {R}_{b} ( \frac{1}{{Z}_{n}} + \frac{1}{{Z}_{m}} )} ] \qquad ( 10 )$$

where I₀(γ^{r}_{c}) and I₁(γ^{r}_{c}) are modified Bessel functions of zero and first order, respectively, and r_{c}, is the radius of a single cell. The solution depends on R_{b}, the resistance between the cells for a unit area, and a defined by

$$α = {R}_{c} \sqrt{\frac{ρ}{h}} , \qquad ( 11 )$$

To calculate the specific resistance for cell-cell and cell-matrix adhesion, the current is assumed to flow radially from under the ventral surface of the cell and the electrode and then escape between cells. A minor amount of current goes directly through the cell membrane by capacitive coupling or transcyptoplasmic shunting. In this model, the total impedance across a cell-covered electrode is composed of the impedance between the ventral surface of the cell and the electrode (related to α), the impedance between cells (indicated by R_{b}), the transcellular impedance (Zₘ), and the impedance of the naked electrode Zₙ. The specific impedances, Zₙ and Zₘ, are frequency dependent. Since Zₙ and Z_{c}. are measured and Zₘ is the impedance of the two cell membranes in series, α, R_{b} and Zₘ (related to Cₘ), are the only adjustable parameters in Eq. 10.

FIG. 5 shows a general circuit diagram for the cellular impedance measuring system. Sensing electrode arrays may be purchased from Applied Biophysics (Troy, N.Y.). Each array consisted of 5 small 10⁻⁴ cm² gold contacts microfabricated on the bottom of 5 separate wells connected to a single larger 10¹ cm² counter electrode. Electrode voltage measurements were made using a Stanford Research SR830 lock-in amplifier that also provided an alternating one volt signal source through a 1 MΩ resistor. The lock-in voltage source impedance, Zₛ, was 50 Ω and the input impedance, Zᵥ, of the phase sensitive detector had resistive, Rᵥ and capacitive, Cᵥ, components of 10 MΩ and 25 pF, respectively. Associated with the source and phase sensitive detector are parasitic lead impedances Zₚₛ, and Zₚᵥ respectively. The electrode impedance of the naked and cell covered electrodes, Zₙ and Z_{c} are assumed functions of frequency. A data acquisition system was developed using a software environment, such as LabVIEW (National Instruments, Austin, Tex.) and interfaced to the SR830 lock-in amplifier.

(a) Numerical Analysis

The model to be fitted in this example is of the form
Z_{c}=Z_{c}(x;a), (12)
where the measured impedance Z_{c}, has both real, R, and imaginary, ℑ, components. The set of independent variables x in this example consist only of the measured frequency, v. The fitting parameter vector, a, has the elements α, R_{b}, and Cₘ. In this experimental system, the noise at different reference frequencies is assumed independent but that the real and imaginary noise components at a given reference frequency can be correlated and have different averages and variances. The X² merit function in this case is

$${χ}^{2} ( α , {R}_{b} , {C}_{m} ) = , {\sum }_{j = 1}^{{N}_{v}} [ ℛ ( {Z}_{cj} - {Z}_{c} ) 𝒥 ( {Z}_{cj} - {Z}_{c} ) ] {Ξ}_{j}^{- 1} [ \frac{ℛ ( {Z}_{cj} - {Z}_{c} )}{𝒥 ( {Z}_{cj} - {Z}_{c} )} ] \qquad ( 13 )$$

where Ξ and Z_{cj} are the covariance matrix and measured impedance at the jth reference frequency. The term Nᵥ, refers to the number of reference frequencies. The matrix Ξⱼ for the jth reference frequency is obtained by repeatedly sampling the data at the jth frequency by using the real and imaginary values to calculate the sample covariance matrix. That is,

$$\begin{matrix}{Ξ}_{j} = [ \begin{matrix}{S}_{j}^{ℛℛ} {S}_{j}^{ℛℛ} \\ {S}_{j}^{𝒥ℛ} {S}_{j}^{𝒥𝒥}\end{matrix} ] , \\ where \\ {S}_{j}^{ℛℛ} = {\sum }_{i = 1}^{N} \frac{ℛ ( {Z}_{cj}^{i} - {\overline{Z}}_{j} ) ℛ ( {Z}_{cj}^{i} - {\overline{Z}}_{j} )}{( N - 1 )} {S}_{j}^{ℛ𝒥} = {\sum }_{i = 1}^{N} \frac{ℛ ( {Z}_{cj}^{i} - {\overline{Z}}_{j} ) 𝒥 ( {Z}_{cj}^{i} - {\overline{Z}}_{j} )}{( N - 1 )} & ( 14 ) \\ {S}_{j}^{𝒥ℛ} = {\sum }_{i = 1}^{N} \frac{𝒥 ( {Z}_{cj}^{i} - {\overline{Z}}_{j} ) ℛ ( {Z}_{cj}^{i} - {\overline{Z}}_{j} )}{( N - 1 )} , and {S}_{j}^{𝒥𝒥} = {\sum }_{i = 1}^{N} \frac{𝒥 ( {Z}_{cj}^{i} - {\overline{Z}}_{j} ) 𝒥 ( {Z}_{cj}^{i} - {\overline{Z}}_{j} )}{( N - 1 )} . & ( 15 )\end{matrix}$$

The averages are calculated from the N data samples at each frequency, i.e.,

$${\overline{Z}}_{j} = \frac{1}{N} {\sum }_{i = 1}^{N} {Z}_{cj}^{i} \qquad ( 16 )$$

while the standard deviations of the real and imaginary impedance components are, respectively, as follows:
Sⱼ^{R}=\sqrt{Sⱼ^{RR}} and Sⱼ^{ℑ}=\sqrt{Sⱼ^{ℑℑ}} (17)
The real and imaginary impedance correlation coefficient can be calculated using the relation

$${r}_{j}^{ℛ𝒥} = {r}_{j}^{𝒥ℛ} = \frac{{S}_{j}^{ℛ𝒥}}{\sqrt{{S}_{j}^{ℛℛ} \sqrt{{S}_{j}^{𝒥𝒥}}}} \qquad ( 18 )$$

If the real and imaginary disturbance terms are written as

$$Δ {Z}_{j} = [ \begin{matrix}Δ {Z}_{j}^{1} \\ Δ {Z}_{j}^{2}\end{matrix} ] = [ \begin{matrix}ℛ ( {Z}_{cj} - {Z}_{c} ) \\ 𝒥 ( {Z}_{cj} - {Z}_{c} )\end{matrix} ] \qquad ( 19 )$$

the Chi² function can be written as

$${χ}^{2} ( a ) = {\sum }_{j = 1}^{{N}_{v}} Δ {Z}_{j}^{T} {Ξ}_{j}^{- 1} Δ {Z}_{j} \qquad ( 20 )$$

The Chi² gradient with respect to the set of parameters a={α, R_{b}, Cₘ}, which will be zero at the Chi² minimum, has components

$$\frac{\partial {χ}^{2} ( a )}{\partial {a}_{k}} = {\sum }_{j = 1}^{{N}_{v}} ( \frac{\partial Δ {Z}_{j}^{T}}{\partial {a}_{k}} {Ξ}_{j}^{- 1} Δ {Z}_{j} + Δ {Z}_{j}^{T} {Ξ}_{j}^{- 1} \frac{\partial Δ {Z}_{j}}{\partial {a}_{k}} ) \qquad ( 21 )$$

Symmetry of the covariance matrix allows Eq. 21 to be simplified where

$$\frac{\partial {χ}^{2} ( a )}{\partial {a}_{k}} = - 2 {\sum }_{j = 1}^{{N}_{v}} ( \frac{\partial Δ {Z}_{c}^{T}}{\partial {a}_{k}} {Ξ}_{j}^{- 1} Δ {Z}_{j} ) \qquad ( 22 )$$

The second partial derivatives of the, Chi² function are given by

$$\frac{{\partial }^{2} {χ}^{2} ( a )}{\partial {a}_{l} \partial {a}_{k}} = {\sum }_{j = 1}^{{N}_{v}} ( \frac{{\partial }^{2} Δ {Z}_{j}^{T}}{\partial {a}_{l} \partial {a}_{k}} {Ξ}_{j}^{- 1} Δ {Z}_{j} + \frac{\partial Δ {Z}_{j}^{T}}{\partial {a}_{k}} {Ξ}_{j}^{- 1} \frac{\partial Δ {Z}_{j}}{\partial {a}_{l}} + \frac{\partial Δ {Z}_{j}^{T}}{\partial {a}_{l}} {Ξ}_{j}^{- 1} \frac{\partial Δ {Z}_{j}}{\partial {a}_{k}} + Δ {Z}_{j}^{T} {Ξ}_{j}^{- 1} \frac{{\partial }^{2} Δ {Z}_{j}}{\partial {a}_{l} \partial {a}_{k}} ) \qquad ( 23 )$$

If the second order partial derivatives are ignored, the curvature or Hessian matrix of the Chi² function is

$$\begin{matrix}\frac{{\partial }^{2} {χ}^{2} ( a )}{\partial {a}_{l} \partial {a}_{k}} = {\sum }_{j = 1}^{{N}_{v}} ( \frac{{\partial }^{2} Δ {Z}_{j}^{T}}{\partial {a}_{k}} {Ξ}_{j}^{- 1} \frac{\partial Δ {Z}_{j}}{\partial {a}_{l}} + \frac{\partial Δ {Z}_{j}^{T}}{\partial {a}_{l}} {Ξ}_{j}^{- 1} \frac{\partial Δ {Z}_{j}}{\partial {a}_{k}} ) \\ or by symmetry & ( 24 ) \\ \frac{{\partial }^{2} {χ}^{2} ( a )}{\partial {a}_{l} \partial {a}_{k}} = 2 {\sum }_{j = 1}^{{N}_{v}} ( \frac{\partial Δ {Z}_{j}^{T}}{\partial {a}_{l}} {Ξ}_{j}^{- 1} \frac{\partial Δ {Z}_{cj}}{\partial {a}_{k}} ) & ( 25 )\end{matrix}$$

For numerical computation, it is convenient to remove the factors of 2 by defining
βₖ=½∂X²(a)/∂aₖ and αₖₗ=½∂²X²(a)/∂aₗ∂aₖ. (26)
The gradient and Hessian are calculated based on the preceding formulation and analyzed using a multi-response Levenberg-Maquardt non-linear optimization. Parameter error estimates from a single fit can be obtained by inverting the Chi² curvature matrix [α] at the Chi² minimum. That is, the matrix C=[α]⁻¹ is the estimated standard error covariance matrix for the fitted parameters a.

3. Results and Discussion

(a) Frequency Dependant Voltage Measurement Statistics

Using a power spectral analysis of the sampled data points at each reference frequency, using different filtering and sampling rates, several sources of noise were identified. Below 200 Hz, harmonic noise at twice the reference frequency dominated the signal. When digital synchronous filtering was used to reduce the harmonic noise to a manageable level, 60 Hz noise became the dominant noise source at reference frequencies below 1 kHz. At these frequencies, the 60 Hertz noise appeared as sum and difference signals of the reference signal following phase sensitive detection. The sum and difference 60 Hz components were aliased to different frequencies when the data sampling rate fell below the Nyquist frequency. Gaussian random noise appeared to be present throughout the measured frequency range. At higher frequencies where filtering effectively reduced the random noise, analog to digital sampling error became the most significant noise source.

FIG. 6 shows the real and imaginary voltage components of a naked electrode and the same cell covered electrode following 22 hours of endothelial cell attachment. Each data point represents the average of 512 voltage measurements sampled at a rate of 32 Hz. The data acquisition time for the entire frequency sweep was under five minutes and the phase sensitive detector low pass filter time constant was set to 30 msec. The low pass filtering time constant was chosen based on a tradeoff between data acquisition time, noise filtering, and a need to acquire a large number of statistically independent data points.

FIG. 7 shows the voltage covariance matrix determinant square root as a function of frequency for the naked and cell covered electrodes. Naked and cell covered voltage noise estimates in both the real and imaginary channels display an overall decreasing trend as a function of increasing frequency. A local maximum, however, occurs in the neighborhood of 60 Hz as a result of power supply noise. At higher frequencies, the cell covered electrode fluctuations tend to exceed those of the naked electrode. In some cases the naked electrode fluctuations fall to insignificant values at high frequencies where the filtering effectively reduces the noise level to the analog to digital noise level. FIG. 7 also shows the stepwise decrease in the analog to digital noise variance as the lock-in amplifier sensitivity is progressively increased to match the decreasing dynamic range of the measured electrode voltage. In an ideal case, analog to digital noise represents a lower bound on the experimental noise that would be obtained if all other forms of noise could be eliminated. The frequency dependent nature of the noise variance underscores the need to include these noise statistics in the numerical optimization.

(b) Voltage Impedance Conversion

To evaluate the α, R_{b} and Cₘ model parameters that characterize the endothelial cell barrier function, it is necessary to convert the voltage measurements into corresponding impedance values. Based on the circuit shown in FIG. 5, a hierarchy of approximations can be used to convert the measured voltages into their equivalent impedance values. In the most general case, the electrode impedance is a function of all the elements shown in FIG. 5, i.e.,

$${Z}_{c} = \frac{{V}_{c} ( {Z}_{s} {Z}_{ps} + {Z}_{cc} {Z}_{s} + {Z}_{cc} {Z}_{ps} ) {Z}_{pv} {Z}_{v}}{{Z}_{pv} {Z}_{v} {Z}_{ps} {V}_{s} - \\ {V}_{c} [ ( {Z}_{s} {Z}_{ps} + {Z}_{cc} {Z}_{s} + {Z}_{cc} {Z}_{ps} ) ( {Z}_{v} + {Z}_{pv} ) + \\ ( {Z}_{s} {Z}_{pv} {Z}_{v} + {Z}_{ps} {Z}_{pv} {Z}_{v} ) ]} \qquad ( 27 )$$

where the impedances and voltages are as defined in FIG. 5. In the limit that the source impedance is very small and the voltmeter impedance is very large, the electrode impedance simplifies to a function of the voltmeter lead parasitic impedance and the 1 MΩ resistor Z_{cc}, i.e.,

$${Z}_{c} = \frac{{V}_{c} {Z}_{cc} {Z}_{pv}}{{V}_{s} {Z}_{pv} - ( {Z}_{cc} {Z}_{pv} )} \qquad ( 28 )$$

If the voltage lead parasitic impedance is very large then the electrode impedance simplifies further to a variation of the classic voltage divider law

$${Z}_{c} = \frac{{V}_{c} {Z}_{cc}}{{V}_{s} - {V}_{c}} \qquad ( 29 )$$

When Z_{cc} is much larger than the Z_{c} then V_{c}<<Vₛ and the constant current assumption,

$${Z}_{c} = \frac{{V}_{c} {Z}_{cc}}{{V}_{s}} \qquad ( 30 )$$

is obtained. The constant current or current clamp, approximation described by Eq. 30 is the approximation used in the ECIS method. However, it is recognized in the art the importance of accounting for lead parasitic impedances and included a balanced impedance equivalent to a 2.21 kΩ resistor and a 4.7 nF capacitor in series to obtain accurate impedance estimates from their voltage measurements.

FIG. 8 summarizes the errors associated with a constant current (Eq. 30), voltage divider (Eq. 29), and a parasitic lead correction (Eq. 28). Each impedance curve in FIG. 8 is normalized to the calculated impedance based on all the circuit elements without any simplifying assumptions (Eq. 27). As FIG. 8 illustrates, the current clamp assumption produces inaccurate estimates of the impedance at both high and low frequencies. The voltage divider approximation improves the impedance estimates at low frequencies but fails at higher frequencies. By including the parasitic resistance and capacitance of the voltmeter leads, a more accurate approximation to the impedance is obtained over the entire frequency range.

(c) Numerical Analysis of α, R_{b} and Cₘ from Impedance Measurements

One problem encountered with using a Levenberg-Marquardt nonlinear optimization algorithm for this particular application is that the parameter estimates repeatedly converged to physically impossible negative values or otherwise diverged catastrophically. To produce physically meaningful estimates of the endothelial cell barrier function parameters α, R_{b} and Cₘ and stabilize the algorithm, it was necessary to constrain the parameter search. Simulated impedance curves were used to set boundaries on a meaningful range of the α, R_{b} and Cₘ parameter values. Calculated parameter steps that produced estimates outside of the range of physically meaningful values were rejected.

Fitting the data to lower frequencies with the same weight as the higher frequencies produced a great deal of instability in α, R_{b} and Cₘ. This was largely attributed to the relatively larger errors that appeared at low frequencies. This problem was corrected by not including the lower frequency points in the optimization. To improve the stability of the algorithm, noise estimates at each frequency were included in the Chi² minimization. Impedance noise estimates at each frequency were obtained by a statistical analysis of multiple voltage samples that had been transformed to their equivalent impedances based on the complete circuit model (Eq. 27). Although including frequency dependent noise in the Chi² minimization greatly improved the stability of the optimization, the algorithm occasionally failed as a result of a singular noise covariance matrix. At high frequencies, where the filtering successfully reduced the noise to the analog to digital level, the noise variance in the real or the imaginary channel would go to zero at times. Including both the analog to digital and electrical noise in the Chi² minimization covariance matrix, however, eliminated these parameter instabilities.

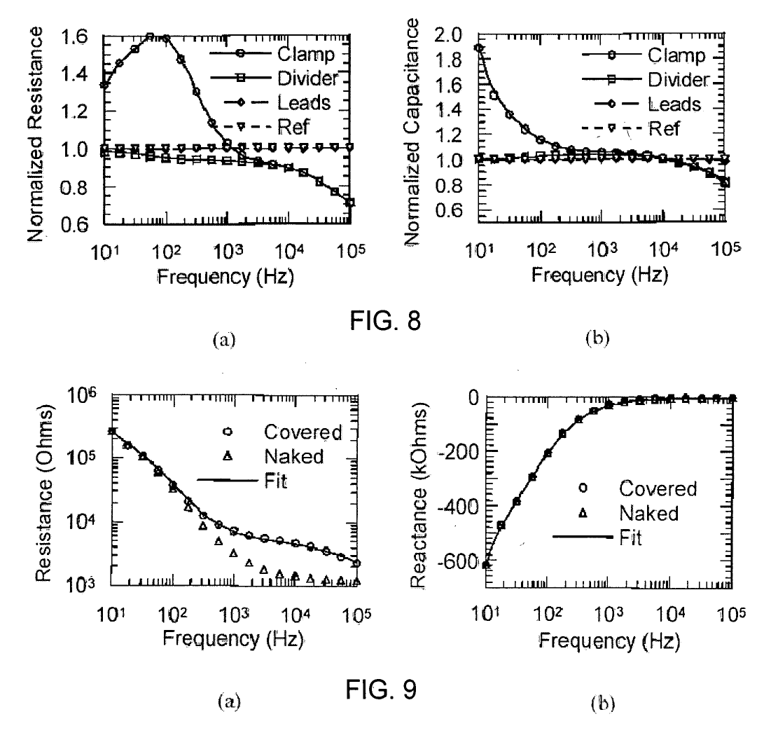

FIG. 9 summarizes the fit to the resistance and reactance using error estimates at each frequency and a voltage to impedance conversion based on the complete circuit analysis. The fitted parameter values were α=4.711±0.001 Ω^{0.5}/cm², R_{b}=1.1080±0.0007 Ω/cm² and Cₘ=1.2396±0.0002 μF/cm ², respectively. The estimated correlation coefficients were α−R_{b}: −0.6996, α−Cₘ: −0.6085 and R_{b}−Cₘ: 0.4567. The optimized fit had a reduced Chi² value of about 2.5272×10⁴. The fact that Chi²>>1 suggests that other sources of error, such as electrode drift, biological fluctuations, or incorrect model assumptions, contributed to the value of Chi². The parameter errors and correlation coefficients were obtained from the estimated covariance matrix found by inverting the Chi² curvature matrix [α] at the minimization point. This and other data consistently showed a pattern of negatively correlated α−R_{b} and α−Cₘ parameters and positively correlated R_{b}−Cₘ parameters.

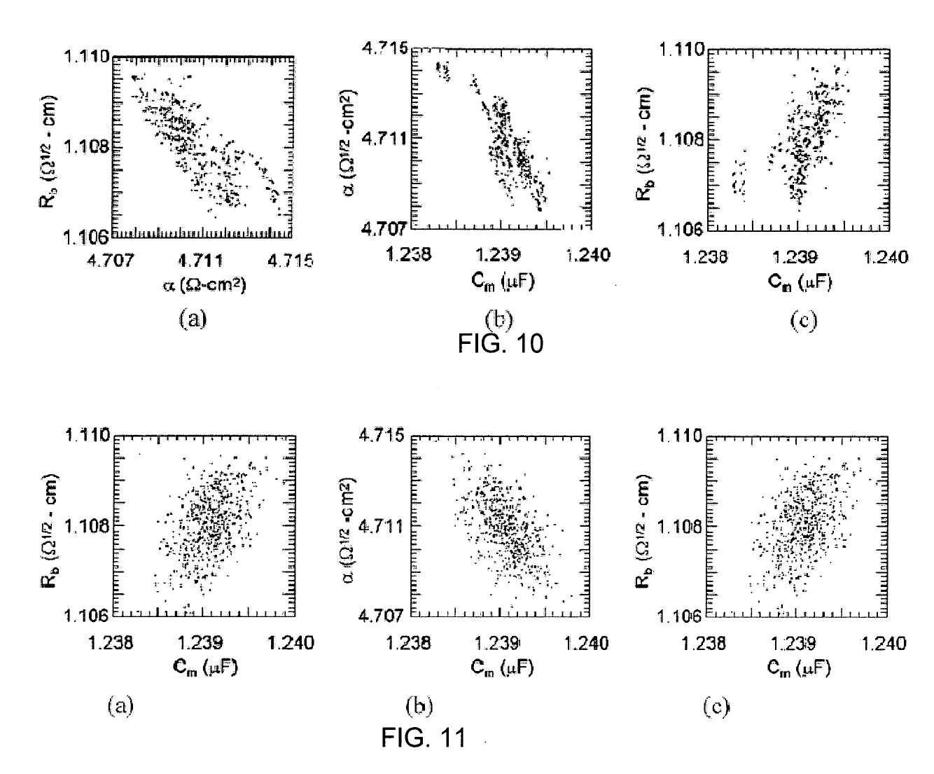

FIG. 10 shows the results of a Monte Carlo simulation using experimental noise added to the average cell covered electrode values. The parameter mean and standard deviations are as follows: α=4.711±0.0010 Ω^{0.5}/cm², R_{b}=1.1080±0.0007 Ω^{0.5}/cm², and Cₘ=1.2396±0.0002 μF/cm². The correlation coefficients are α−R_{b}: −0.7655, α−Cₘ: −0.8141 and R_{b}Cₘ: 0.6096, and the goodness of fit Chi²=2.5277×10⁴. These error and correlation estimates, based on the statistics of the Monte Carlo simulation results, are consistent with the results of a single fit using the Chi² curvature matrix.

The assumed Gaussian nature of the experimental noise in the least squares algorithms used in this example is an important consideration. The multidimensional least squares minimization that the multi-response Levenberg-Marquardt algorithm produces is equivalent to a maximum likelihood estimate if the noise distribution is Gaussian. The experimental noise distribution in this example, however, is inherently non-Gaussian in nature. To ensure that the non-Gaussian nature of the experimental noise did not produce erroneous results, two-dimensional Gaussian noise with the same statistics as the experimental noise was generated and added to the average cell covered electrode data and subsequently fit. FIG. 11 shows the equivalent Monte Carlo simulation using generated 2D Gaussian noise with the same covariance matrix as the cell covered electrode experimental noise. The discrepancies between the experimental and generated Gaussian noise Monte Carlo simulation can be partially explained by the deterministic components of the experimental noise. FIG. 11 shows that two-dimensional Gaussian noise with the same frequency dependent covariance matrix as the experimental noise produces comparable parameter error estimates.

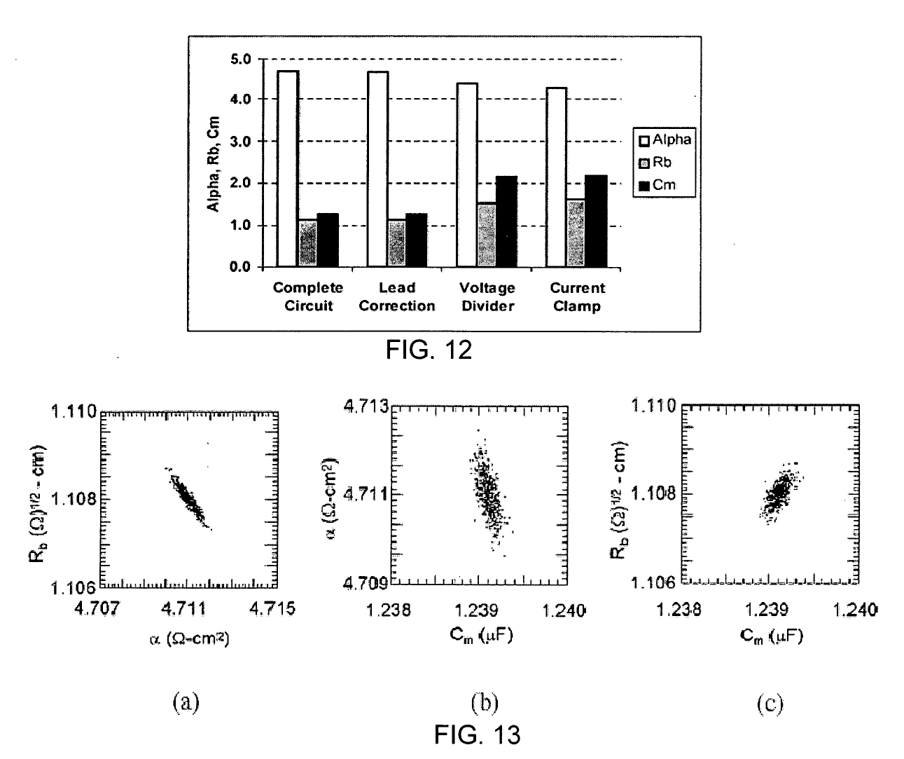

The voltage to impedance conversion method can introduce systematic errors in the calculated barrier function parameters, α, R_{b} and Cₘ. FIG. 12 illustrates the pattern that results from analyzing a cell covered electrode using the constant current assumption, a voltage divider estimate, a correction for the parasitic impedance of the voltage measuring leads, and a complete circuit analysis that includes the input and source impedance of the lock-in amplifier. This same systematic trend in the α, R_{b} and Cₘ parameters was observed consistently when different cell covered electrodes were measured. The approximations that account for the voltage divider effect, lead parasitic impedances, and instrumental impedances correspond to approximations that include increasing degrees of circuit loading. With increasing loading, a increases and R_{b} decreases. The parameter Cₘ is influenced most strongly by including the parasitic impedance of the voltage leads. The same trend is observed when data is simulated with similar barrier function parameters and analyzed using the different circuit model approximations.

Systematic and random electrical noise contributes to the parameter uncertainty not only through the cell covered impedance, Z_{c} but also through measurement errors in the naked electrode impedance, Zₙ. Table 1 below summarizes a series of Monte Carlo simulations where experimentally measured noise is added to only the naked electrode, to only the cell covered electrode, and to both the naked and cell covered electrode. These Monte Carlo simulations were based on a series of 512 data sets that were obtained by adding the experimental noise to the average impedance values. The off diagonal values indicate the correlation coefficients while the diagonal error estimates represent the standard deviation of the ensemble. Experimental noise, characteristic of each frequency, was added to the simulated data sets and subsequently analyzed to obtain parameter values. The results of these simulations indicate that noise in both the naked electrode and the cell covered impedance estimates contribute to the parameter error bounds.

TABLE 1
Monte Carlo parameter error estimates produced by noise propagation
through the naked and cell covered electrode data

| | | α | R_{b} | Cₘ |
|---|---|---|---|---|
| Naked | α | 4.711 ± 0.002 | −0.3073 | −0.9919 |
| Electrode only | R_{b} | | 1.1080 ± 0.0002 | 0.3564 |
| (Chi² = 2.526 × 10⁴) | Cₘ | | | 1.2396 ± 0.0004 |
| Cell covered | α | 4.711 ± 0.001 | −0.7655 | −0.8141 |
| Electrode only | R_{b} | | 1.1080 ± 0.0007 | 0.6096 |
| (Chi² = 2.528 × 10⁴) | Cₘ | | | 1.2396 ± 0.0002 |
| Naked and cell | α | 4.711 ± 0.003 | −0.4147 | −0.9272 |
| Covered electrode | R_{b} | | 1.1080 ± 0.0008 | 0.3760 |
| (Chi² = 2.526 × 10⁴) | Cₘ | | | 1.2396 ± 0.0004 |

Table 2 summarizes the results of a Monte Carlo simulation obtained using two-dimensional Gaussian noise and simulated cell covered impedance data. A series of 512 data sets were simulated with the model parameters values α=4.711 Ω^{0.5}/cm², R_{b}=1.1080 Ω/cm², and Cₘ=1.2396 μF/cm². Two dimensional Gaussian noise having the same covariance matrix as the corresponding experimentally measured noise was generated at each frequency and added to either the naked, cell covered, or both the naked and cell covered impedances. When two-dimensional Gaussian noise is added to only the simulated cell covered electrode data and analyzed, errors comparable to those obtained during the analysis of experimental data were obtained. The reduced chi squared parameter, however, is on the order of unity. This helps to validate the numerical optimization algorithm and suggests that other factors, such as electrode drift or model limitations, is contributing to the large Chi² values. These Monte Carlo simulations also illustrate that a significant contribution to the parameter errors is produced by the propagation of noise through the naked electrode measurements.

TABLE 2
Monte Carlo parameter error estimates produced by the propagation of two-dimensional
Gaussian* noise through the naked and cell covered electrode data

| | | α | R_{b} | Cₘ |
|---|---|---|---|---|
| Naked** | α | 4.711 ± 0.002 | −0.6967 | −0.8191 |
| Electrode only | R_{b} | | 1.1080 ± 0.0005 | 0.7019 |
| (Chi² = 5.1710) | Cₘ | | | 1.2396 ± 0.0004 |
| Cell covered | α | 4.711 ± 0.001 | −0.7124 | −0.6218 |
| Electrode only | R_{b} | | 1.1080 ± 0.0007 | 0.4751 |
| (Chi² = 0.9977) | Cₘ | | | 1.2396 ± 0.0002 |
| Naked and cell | α | 4.711 ± 0.003 | −0.6458 | −0.7440 |
| Covered electrode | R_{b} | | 1.1080 ± 0.0009 | 0.5287 |
| (Chi² = 6.1871) | Cₘ | | | 1.2396 ± 0.0003 |

It is noted that the frequency dependent two-dimensional Gaussian noise with the same covariance matrix as the experimental data was generated and added to simulated cell covered electrode data. Also, frequency dependent noise based on the cell covered electrode was used in the optimization in all three cases shown in Table 2.

The multi-response Levenberg-Maquardt algorithm optimizes a fit to both the real and the imaginary parts of the impedance. Table 3 summarizes the results of fitting to only the real, only to the imaginary, to the magnitude, and to both the real and the imaginary data. Compared to a conventional Levenberg-Marquardt algorithm, where only one dimensional response data can be analyzed, the parameter error estimates and correlation coefficients are smaller. Error estimates based on the Chi² curvature from a single measurement and the results of Monte Carlo simulations both indicated that the parameter error estimates are smaller using both the real and the imaginary data. Even if the magnitude data is used in a conventional single response Levenberg-Marquardt algorithm, a multi-response Levenberg-Marquardt algorithm still produces more stable α, R_{b} and Cₘ parameter estimates. In addition, the degree of correlation between the parameters is reduced when both real and imaginary data are used in a multi-response algorithm. The smaller and more stable parameter estimates, however, are obtained at the expense of the added complexity of the noise sampling and numerical implementation of the algorithm.

TABLE 3
Comparison of optimized parameter error estimates using only real,
only imaginary, only magnitude, and both real and imaginary data

| | | α | R_{b} | Cₘ |
|---|---|---|---|---|
| Real and Imag | α | 4.711 ± 0.002 | −0.6996 | −0.6085 |
| Optimization | R_{b} | | 1.1080 ± 0.0007 | 0.4567 |
| (Chi² = 2.527 × 10⁴) | Cₘ | | | 1.2396 ± 0.0002 |
| Real | α | 5.272 ± 0.005 | −0.9410 | −0.7245 |
| Optimization | R_{b} | | 0.805 ± 0.004 | 0.7314 |
| (Chi² = 9.568 × 10²) | Cₘ | | | 1.23811 ± 0.0004 |
| Imag | α | 4.219 ± 0.005 | −0.9530 | −0.8302 |
| Optimization | R_{b} | | 1.282 ± 0.005 | 0.8256 |
| (Chi² = 6.319 × 10²) | Cₘ | | | 2.146 ± 0.003 |
| Magnitude | α | 4.706 ± 0.006 | −0.9521 | −0.7266 |
| Optimization | R_{b} | | 1.300 ± 0.006 | 0.7816 |
| (Chi² = 1.946 × 10²) | Cₘ | | | 1.477 ± 0.001 |

Increasing the filter time constant reduces the errors in the three parameters. Longer filter time constants, however, require increased data acquisition times. If longer filter time constants are used and sufficient time is not allowed for the instrument to settle as it is switched to the next highest sampling frequency, then α and R_{b} are systematically overestimated and Cₘ is systematically underestimated. Changes in cellular barrier 10 function must also be negligible over the data acquisition times. Sufficiently increasing the filtering time constant effectively reduces the noise to the level of the analog to digital sampling interval error. The filter time constants required to reduce noise fluctuations at low frequencies to the level of analog to digital, however, mask biologically relevant fluctuations.

FIG. 13 summarizes the results of an analog to digital noise Monte Carlo simulation performed using the experimentally representative set of α, R_{b} and Cₘ parameter values: α=4.711 Ω^{0.5}/cm², R_{b}=1.108 Ω/cm² and Cₘ=1.2396 μF/cm². Frequency dependent uniform random noise, based on the digitization intervals of the lock-in amplifier, was transformed to an equivalent impedance noise using the complete circuit model shown in FIG. 6 and added to the modeled naked and cell covered impedance values. The two dimensional uniform random noise was assumed uncorrelated. The results of evaluating 512 simulated data sets using a Multi-response Levenberg-Marquardt gave the following results: α=4.7110±0.0003 Ω^{0.5}/cm², R_{b}=1.1080±0.0002 Ω/cm² and Cₘ=1.2396±0.00008 μF/cm². The correlation coefficients were α−R_{b}=−0.9480, α−Cₘ: −0.6166 and R_{b}−Cₘ: 0.6455. Frequency dependent uniform random noise, characteristic of the analog to digital converter sampling intervals, contributed to both the parameter error bounds and the Chi² values through both the naked, Zₙ and cell covered, Z_{c} impedance measurements. The optimized fit had a Chi²ᵥ=2.0442.

4. Conclusions

A progressive systematic error reduction in the estimated electrode impedance and barrier function parameter estimates were obtained by converting the measured voltages using a constant current assumption, a voltage divider assumption, and a correction for parasitic resistance and capacitance in the lock-in amplifier leads. Greater stability and smaller parameter errors were obtained by using a multi-response Levenberg-Marquardt algorithm with the parameters constrained within physically meaningful values. Noise statistics were incorporated into the non-linear fit due to the large range in noise variance over the measured frequencies. Noise in the measured naked electrode measurements produced significant contributions to the parameter error estimates. The very large reduced Chi² values, obtained from fits to experimental data, suggest that biological fluctuations or model limitations should be considered in future work. Including both real and imaginary impedance data significantly improved the stability of the parameter fits in this example. The three parameters α, R_{b} and Cₘ were consistently correlated with each other.

### Example 2

### Modeling Error and Stability of Endothelial Cytoskeletal-Membrane Parameters Based on Modeling Transendothelial Impedance as Resistor and Capacitor in Series

Activation of signal transduction pathways and remodeling of endothelial cell-cell and cell-matrix adhesion are steps that can regulate, for example, inflammatory edema, wound injury and repair, and angiogenesis. Inflammatory edema formation, for example, may be characterized by dynamic changes in endothelial cell-to-cell and cell-matrix attachment, which regulates the proper balance of fluid and protein between intravascular and interstitial compartments. Endothelial barrier function relies on the mechanical properties of the cytoskeleton and its mechanical connection to the cell's membrane. Thus, quantification of endothelial cytoskeletal-membrane properties may be critical to precisely evaluate the mechanisms that regulate endothelial barrier function. By measuring transcellular impedance across a confluent monolayer inoculated on a microelectrode, inflammatory stimuli like histamine and thrombin may regulate human endothelial barrier function in a rapid, non-linear, and time-dependent fashion. Since the measured transendothelial impedance may be dependent on endothelial cell-cell and cell-matrix adhesion, transendothelial impedance can be used to quantify cell-membrane properties.

Identifying the specific cell adhesion sites and cytoskeletal-membrane properties that regulate membrane integrity and function under physiological and pathological conditions can represent a complex task due to intervening cytoskeletal network mechanically couples cell-cell and cell-matrix adhesion sites. For example, if the cytoskeleton is viewed as an integrative structure, external stimuli could disrupt cell-cell adhesion through two basic mechanisms. Activation of signal transduction events could decrease adhesion at cell-matrix sites and cause cell rounding, which in turn, could result in a secondary or reactive loss in cell-cell adhesion. Alternatively, activation of signal transduction pathways may directly target cell-cell adhesion sites and cause a direct loss in cell-cell adhesion with a reactive loss in cell-matrix adhesion. Because there are distinct adhesion proteins at cell-cell and cell-matrix sites that could be differentially affected by signal transduction pathways, it may be necessary to identify the spatial and temporal characteristics by which molecular signals differentially affect cytoskeletal-membrane properties. Thus, numerical models and simulations are a critical part of evaluating the complexities of these signal transduction pathways.

A closed-form mathematical model proposed in the Proceedings of the National Academy of Science characterizes cell-cell and cell-matrix adhesion in cultured fibroblasts by measuring transcellular impedance of a cultured monolayer grown on a microelectrode exposed to an alternating current. (Giaever et al., 1991, “Giaever”). Measurements of cell-cell and cell-matrix adhesion can be resolved in confluent monolayers by mathematically modeling the impedance across a cell-covered electrode as an electrical circuit consisting of a capacitor and resistor in series. A closed-form solution can be based on treating cells as disk shape and organized in a fashion in which individual cells make contact with its neighbor but with gaps between cells (e.g., FIG. 1).

The boundary geometry and conditions of the Giaever model were not disclosed in this report or in a later one published by the same authors in cultured epithelial cells. The model can characterizes transcellular impedance into three unknown solution parameters in confluent cultured cells: R_{b} (the impedance due to cell-cell adhesion); α or alpha (the impedance due to cell-matrix adhesion); and Cₘ (membrane capacitance due to transcellular electrical conduction).

In principle, the unknown parameter solutions of cell surface membrane properties are resolvable provided there are at least an equal number of experimental measurements for the number of unknown solution parameters. One advantage of measuring barrier function using an alternating current, rather than the conventional use of a direct current, is that experimental impedance is measured at multiple frequencies, which permits simulating and solving for these unknown membrane properties.

The Giaever model was demonstrated with molecular and microscopic approaches by the inventors' laboratory in a report on cultured endothelial cells stimulated with histamine. (Moy et al., 2000). The inventors reported that histamine disrupted human endothelial barrier function by primarily disrupting cell-cell adhesion, while the restoration of barrier function was dependent on some yet unidentified interaction at cell-matrix sites. Changes in R_{b} correlated with low submicron displacements in cell-cell contact when measured with transmission but not scanning electron microscopy.

Example 2 demonstrates that edemagenic stimuli alter endothelial cytoskeletal-membrane function at a level that cannot be detected by light microscopic techniques. The inventors also report that thrombin transiently disrupted barrier function through a similar paradigm as histamine with the exception that thrombin mediated a greater and more sustained loss in endothelial barrier function, in part, through contractile-dependent disruption of cell-cell adhesion. Along this same line of evidence, a comprehensive study in which biophysical and numerical approaches were integrated with microscopic and biochemical approaches to evaluate how phorbol esters and thrombin regulate porcine pulmonary artery endothelial barrier function through actin-dependent mechanical forces was conducted. Like the histamine responses reported in cultured human endothelial cells, thrombin-mediated changes in transendothelial resistance occurred without microscopic changes in gap formation in living cells when viewed with time-lapsed microscopy. Taken together, these data reinforce the need for new technical approaches that breakdown endothelial barrier function into separate indices of cell-cell and cell-matrix adhesion.

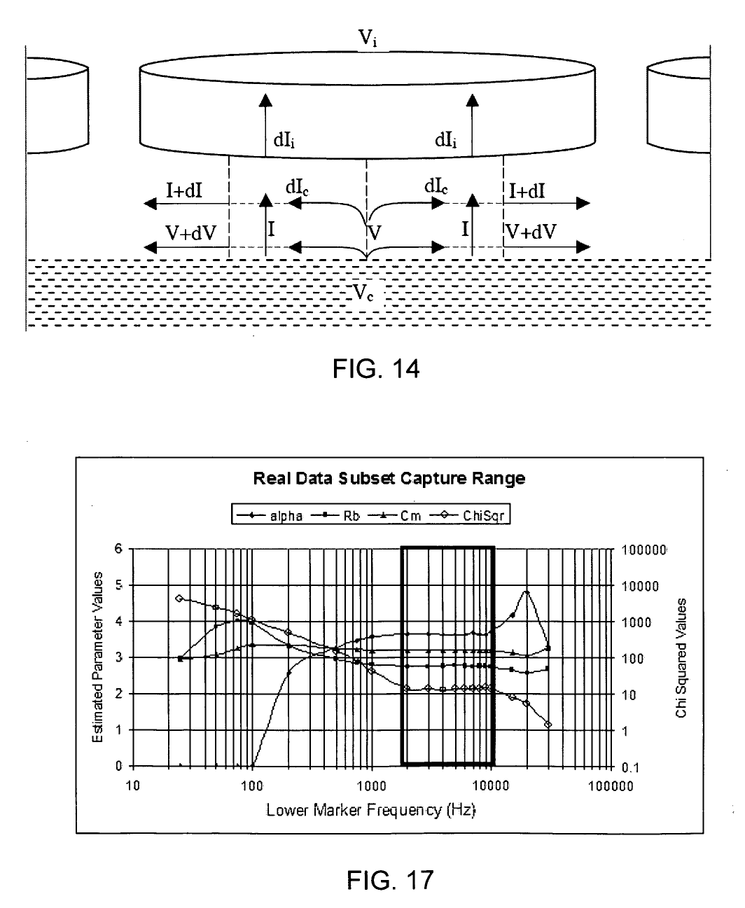

It is noted that the stability, predictability and reliability of the numerical model greatly depend on a thorough understanding of the numerical algorithms, the limitation of model assumptions, and the experimental factors that create model bias and instability. Without understanding these systematic factors that affect the model, the solution parameters of cell membrane properties could be erroneous and lead to misinterpretation. In this example, a complete description of the boundary conditions and intermediate algorithms that result in the closed-form mathematical model of transendothelial impedance is presented based on the following assumptions:
(1) that a cultured monolayer is treated as organized disk shape arranged in an ordered pattern (FIG. 14); and
(2) the impedance across a cell-covered electrode is treated as a resistor and capacitor in series.
   By calculating a Chi-Square (X²) and using a multi-response Levenberg Marquardt, non-linear optimization model of the real only, imaginary only, real and imaginary (in complex form) and real and imaginary (in magnitude form) data, one derives further insight into modeling error and stability of the model's solution parameters.
   Based on these analyses, the following issues are addressed in Example 2:
(1) How much modeling error exists between optimization procedures and visual curve fitting;
(2) How much modeling error and instability exists between experimental and calculated measurements;
(3) Is modeling stability and error unique and a function of frequency for real and imaginary data;
(4) What numerical approach is required to derive stable and reproducible solution parameters in the real and imaginary data; and
(5) Is a model based on a repeating disk pattern and a resistor and capacitor in series sufficient in characterizing the entire impedance data spectrum.

1. Materials and Methods

Cultured human umbilical vein endothelial cells (HUVEC) were prepared by collagenase treatment of freshly obtained human umbilical veins. Harvested primary cultures designated for cell-adhesion assays were plated on 60-mm tissue culture plates that were coated with 100 ug/ml of fibronectin (Collaborative Research Inc., Bedford, Mass.). Experiments were conducted after cultures reached 2-d postconfluency. All cells were cultured in Medium 199 and supplemented with 20% heat-inactivated fetal calf serum, basal medium Eagle vitamins and amino acids, glucose (5 mM), glutamine (2 mM), penicillin (100 U/ml), and streptomycin (100 ug/ml). Cultures were identified as endothelial cells by their characteristic uniform morphology, uptake of acetylated low-density lipoproteins, and indirect immunofluorescent staining for factor VIII.

2. Measurement of Transendothelial Impedance on a Microelectrode Biosensor

Endothelial barrier function was measured using an electrical substrate impedance sensing (ECIS) technique. In this technique, cells were cultured on a small gold electrode (5×10⁻⁴ cm²) using culture medium as the electrolyte, and barrier function was measured dynamically by determining the electrical impedance of a cell-covered electrode. A variable voltage, alternating signal was supplied through a 1-MΩ resistor between frequencies of 25 to 60,000 Hz. Voltage and phase data were measured with a model SRS830 lock-in amplifier (Stanford Research Systems, Sunnyvale, Calif.) stored and processed with a personal computer. The same computer also controlled the output of the amplifier and mechanical relay switches to different electrodes using custom software written by Applied Biophysics Inc. (Troy, N.Y.). Cultured HUVEC's were inoculated on electrodes at a confluent density of 10⁵ cells/cm². Algorithms mathematically converted the in-phase and the out-of-phase voltage into the resistance and capacitance respectively based on the assumption that both the naked electrode and the cell monolayer are treated as resistor and capacitor in series.

3. Software Architecture of the Numerical Modeling

LabVIEW 6.0 graphical application development environment for data acquisition, analysis, signal processing, and instrument control was obtained from National Instruments (Austin, Tex.). Microsoft Visual Studio integrated development environment (IDE) was obtained from Microsoft (Redmond, Wash.). LabView algorithms called Electrical Impedance Modeling Analysis and Simulation (EMAS) were developed to model cell membrane parameters from the measured transendothelial impedance as discussed in the following section.

4. Modeling Approach

Transendothelial impedance across a cell-covered electrode was measured at 23 different frequencies. FIG. 14 represents a diagram of the primary current flow paths across a confluent monolayer. Each current flow path is affected by small spatial changes in the cellular shape, which dynamically modifies transendothelial impedance at each of the measured frequencies. Voltage spreads both horizontally in a radial fashion from the cells center as well as vertically through the cells membrane. V_{c} is the voltage present at the surface of the electrode, V is the voltage present in the space between cells ventral surface and the electrode surface, and Vi is the voltage along the apical surface of the cell. A change in voltage, dV, occurs as the current I spreads horizontally in the space between cells ventral surface and the electrode surface. Current dI_{c} flows vertically from the electrode surface where a change in current, dIᵢ, crosses vertically across two cell membranes. Finally, a change in current, dI, occurs as current spreads horizontally in the space between cells ventral surface and the electrode surface.

Three separate cardinal current flow paths govern the total modeled impedance across a confluent monolayer of endothelial cells. The first current flow path lies between the ventral surface of the monolayer and the surface of the naked electrode and is describe by the parameter alpha (α), which is expressed in units of \sqrt{Ω}·cm. The α term is defined by the Eq. 11, which is dependent on the average separation distance (h) between the ventral membrane surface and the substratum, the solution resistivity, rho (ρ), of the culture medium, and the cell radius (R_{c}). The current flow path between the adjacent edges of the cells within the monolayer is labeled with the parameter (R_{b}) and is expressed in units of Ω·cm². The final current flow path is capacitive in nature and relates to trans-cellular current flow through a ventral and dorsal plasma membrane. The trans-cellular current is dominated by the membrane capacitance parameter (Cₘ) along with a trans-membrane resistance and a trans-cytoplasmic component, which is fixed within the model. The parameter, Cₘ is reported in μF/cm².

5. The Modeling Equations

The following model characterizes endothelial cells as disk shape and arranged in a repeating pattern. If r_{c} is the defined as the cell radius, then the cell area is π×r_{c}² and the cell perimeter is 2×π×r_{c}. The general model predicts the experimental impedance spectrum by applying classical Ohm and Kirchoff laws for electrical currents. Eq. 31 through 33 are Ohms law formulations and Eq. 34 applies Kirchoff's current law, which couples Eqs. 31 through 33.

$$\begin{matrix}- dV = \frac{1}{h \cdot 2 π r} ρ Idr & ( 31 ) \\ {V}_{c} - V = \frac{{Z}_{n}}{2 π r \cdot dr} {dI}_{c} & ( 32 ) \\ V - {V}_{i} = \frac{{Z}_{m}}{2 π r \cdot dr} {dI}_{i} & ( 33 ) \\ dI = {dI}_{c} - {dI}_{i} & ( 34 )\end{matrix}$$

For Eqs. 31 through 34 above, V_{c} is the voltage present at the surface of the electrode; V is the voltage present in the space between cells ventral surface and the electrode surface, and Vᵢ is the voltage along the apical surface of the cell. A change in voltage, dV, occurs as current, I, spreads horizontally in the space between cells ventral surface and the electrode surface. Current dI_{c} flows vertically from the electrode surface. A change in current occurs when a part of the current dIᵢ crosses vertically across two cell membrane surfaces and a part dI spreads horizontally in the space between the cell ventral surface and the electrode surface. The inventors assume that the electrode voltage potential is the same at any arbitrarily chosen radius from the center of the disk-shape cell. Eq. 31 describes the horizontal drop in voltage in the ventral space below the cell as it spreads radially from the center of the cell towards its outer edges. This horizontal voltage drop is governed by ρ (rho) and h (height), which have been previously defined. Eq. 32 describes the vertical change in voltage from the electrode surface into the medium below the ventral surface of the cell. These voltages are labeled V_{c} (the voltage of the electrode) and V (the voltage associated with the cells ventral surface) respectively in FIG. 14. Eq. 33 describes the change in voltage from the ventral to apical surfaces of the cell membrane and adds the voltage labeled Vᵢ above the cells apical surface in FIG. 14. The Eq. 34 couples the Eqs. 31-33 using Kirchoff's current law by the current paths traversed from one electrode to the other. The current makes a fork and splits in two directions. One path is coupled capacitively across the cell membrane and the other path travels in the paracellular compartments below and between the cells involved. The above equations lead to the following result where \dot{V} and \ddot{V} are the first and second derivatives of the voltage at the cell ventral surface with respect to the radius respectively. Also Zₙ and Zₘ are the impedances associated with the naked electrode and cell membrane respectively. The second derivative may be expressed as follows:

$$- \ddot{V} = \frac{1}{r} \dot{V} - {\underbrace{\frac{ρ}{h} ( \frac{1}{{Z}_{n}} + \frac{1}{{Z}_{m}} )}}_{γ} V + {\underbrace{\frac{ρ}{h} ( \frac{{V}_{c}}{{Z}_{n}} + \frac{{V}_{i}}{{Z}_{m}} )}}_{β} \qquad ( 35 )$$

which is in the following form

$$\ddot{V} + \frac{1}{r} \dot{V} - {γ}^{2} V + β = 0 \qquad ( 36 )$$

and has the following solution

$$V ( r ) = {AI}_{0} ( γ r ) + {BK}_{0} ( γ r ) + \frac{β}{{γ}^{2}} . \qquad ( 37 )$$

Since K₀(γr)→∞ as r→0, B=0 and thus

$$V ( r ) = {AI}_{0} ( γ r ) + \frac{β}{{γ}^{2}} = {AI}_{0} ( γ r ) + \frac{{Z}_{m}}{{Z}_{n} + {Z}_{m}} {V}_{c} + \frac{{Z}_{n}}{{Z}_{n} + {Z}_{m}} {V}_{i} . \qquad ( 38 )$$

The constant A depends on the boundary conditions. The radius is r_{c} and Vᵢ=0 implies

$$⇒ \frac{β}{{γ}^{2}} = \frac{{Z}_{m}}{{Z}_{n} + {Z}_{m}} {V}_{c} . \qquad ( 39 )$$

The total current I(r) at r=r_{c} is given by

$$\begin{matrix}I ( {r}_{c} ) = {I}_{ct} - {I}_{it} = {\int }_{0}^{{r}_{c}} ⅆ {I}_{c} - {\int }_{0}^{{r}_{c}} ⅆ {I}_{i} \\ where & ( 40 ) \\ {I}_{it} = {\int }_{0}^{{r}_{c}} \frac{2 π r}{{Z}_{m}} V ⅆ r = \frac{2 π}{{Z}_{m}} {\int }_{0}^{{r}_{c}} [ {ArI}_{0} ( γ r ) + \frac{{Z}_{m}}{{Z}_{n} + {Z}_{m}} {V}_{c} r ] ⅆ r \\ implies ⇒ \frac{2 π {r}_{c}^{2} A}{{Z}_{m} γ {r}_{c}} {I}_{1} ( γ {r}_{c} ) + \frac{π {r}_{c}^{2} {V}_{c}}{{Z}_{n} + {Z}_{m}} \\ and & ( 41 ) \\ {I}_{ct} = {\int }_{0}^{{r}_{c}} \frac{2 π r}{{Z}_{n}} ( {V}_{c} - V ) ⅆ r = \frac{π {r}_{c}^{2}}{{Z}_{n}} {V}_{c} - \frac{2 π}{{Z}_{n}} {\int }_{0}^{{r}_{c}} [ {ArI}_{0} ( γ r ) + \frac{{Z}_{m}}{{Z}_{n} + {Z}_{m}} {V}_{c} r ] ⅆ r \\ also implies \\ ⇒ \frac{π {r}_{c}^{2}}{{Z}_{n}} {V}_{c} - \frac{2 π {r}_{c}^{2} A}{{Z}_{n} γ {r}_{c}} {I}_{1} ( γ {r}_{c} ) - \frac{π {r}_{c}^{2}}{{Z}_{n}} \frac{{Z}_{m}}{{Z}_{n} + {Z}_{m}} {V}_{c} = \frac{2 π {r}_{c}^{2} A}{{Z}_{n} γ {r}_{c}} {I}_{1} ( γ {r}_{c} ) + \frac{π {r}_{c}^{2}}{{Z}_{n} + {Z}_{m}} V . & ( 42 )\end{matrix}$$

Therefore the total current I(r) at r=r_{c} is obtained as

$$I ( {r}_{c} ) = {I}_{ct} - {I}_{it} = - \frac{2 π {r}_{c}^{2} A}{{Z}_{n} γ {r}_{c}} {I}_{1} ( γ {r}_{c} ) ( \frac{1}{{Z}_{n}} + \frac{1}{{Z}_{m}} ) . \qquad ( 43 )$$

Coefficient A can be determined as follows. In one respect,

$$V ( {r}_{c} ) = {AI}_{0} ( γ {r}_{c} ) + \frac{{Z}_{m}}{{Z}_{n} + {Z}_{m}} {V}_{c} . \qquad ( 44 )$$

In other respects, from the boundary condition,

$$\begin{matrix}\begin{matrix}V ( {r}_{c} ) = {\underbrace{I ( {r}_{c} ) \frac{{R}_{b}}{π {r}_{c}^{2}}}}_{Boundary Condition} \\ = \frac{2 π {r}_{c}^{2}}{γ {r}_{c}} {AI}_{1} ( γ {r}_{c} ) ( \frac{1}{{Z}_{n}} + \frac{1}{{Z}_{m}} ) \frac{{R}_{b}}{π {r}_{c}^{2}} \\ = \frac{2}{γ {r}_{c}} {AI}_{1} ( γ {r}_{c} ) ( \frac{1}{{Z}_{n}} + \frac{1}{{Z}_{m}} ) {R}_{b} \\ = A [ {I}_{0} ( γ {r}_{c} ) + \frac{2}{γ {r}_{c}} {I}_{1} ( γ {r}_{c} ) ( \frac{1}{{Z}_{n}} + \frac{1}{{Z}_{m}} ) {R}_{b} ] \\ = \frac{1}{{Z}_{n} + {Z}_{m}} {V}_{c}\end{matrix} \\ which implies that & \\ ⇒ A = \frac{\frac{- {Z}_{m}}{{Z}_{n} + {Z}_{m}} {V}_{c}}{{I}_{0} ( γ {r}_{c} ) + \frac{2}{γ {r}_{c}} {I}_{1} ( γ {r}_{c} ) ( \frac{1}{{Z}_{n}} + \frac{1}{{Z}_{m}} ) {R}_{b}} . & ( 45 )\end{matrix}$$

Now, the equivalent impedance is given by the following

$$\frac{{Z}_{c}}{π {r}_{c}^{1}} = \frac{{V}_{c} - {V}_{i}}{{I}_{ct}} ⇒ \frac{1}{{Z}_{c}} = \frac{1}{π {r}_{c}^{2}} \frac{{I}_{ct}}{{V}_{c}} = \frac{1}{π {r}_{c}^{2}} \frac{1}{{V}_{c}} \{ - \frac{2 π {r}_{c}^{2}}{{Z}_{n} γ {r}_{c}} {I}_{1} ( γ {r}_{c} ) \frac{\frac{- {Z}_{m}}{{Z}_{n} + {Z}_{m}} {V}_{c}}{{I}_{0} ( γ {r}_{c} ) + \frac{2}{γ {r}_{c}} {I}_{1} ( γ {r}_{c} ) ( \frac{1}{{Z}_{n}} + \frac{1}{{Z}_{m}} ) {R}_{b}} + \frac{π {r}_{c}^{2}}{{Z}_{n} + {Z}_{m}} {V}_{c} \} = \frac{1}{{Z}_{n}} \{ \frac{{Z}_{n}}{{Z}_{n} + {Z}_{m}} + \frac{\frac{{Z}_{m}}{{Z}_{n} + {Z}_{m}}}{\frac{γ {r}_{c}}{2} \frac{{I}_{0} ( γ {r}_{c} )}{{I}_{1} ( γ {r}_{c} )} + ( \frac{1}{{Z}_{n}} + \frac{1}{{Z}_{m}} ) {R}_{b}} \} . \qquad ( 46 )$$

By letting

$$γ {r}_{c} = α \sqrt{\frac{1}{{Z}_{n}} + \frac{1}{{Z}_{m}}} = \sqrt{\frac{ρ}{h} ( \frac{1}{{Z}_{n}} + \frac{1}{{Z}_{m}} )} , \qquad ( 47 )$$

the closed form solution is

$$\frac{1}{{Z}_{c}} = \frac{1}{{Z}_{n}} ( \frac{{Z}_{n}}{{Z}_{n} + {Z}_{m}} + \frac{\frac{{Z}_{m}}{{Z}_{n} + {Z}_{m}}}{\frac{α \sqrt{\frac{1}{{Z}_{n}} + \frac{1}{{Z}_{m}}}}{2} \frac{{I}_{0} ( α \sqrt{\frac{1}{{Z}_{n}} + \frac{1}{{Z}_{m}}} )}{{I}_{1} ( α \sqrt{\frac{1}{{Z}_{n}} + \frac{1}{{Z}_{m}}} )} + {R}_{b} ( \frac{1}{{Z}_{n}} + \frac{1}{{Z}_{m}} )} ) . \qquad ( 48 )$$

The symbols I₀(γr_{c}) and I₁(γr_{c}) represent Bessel functions of the first kind, order zero and one, respectively with arguments of γr_{c}. In the formulation for γr_{c}, the parameter for α, which may represent vascular attachment is shown with alternate relations exposing h and ρ. It is noted that the impedance due to transcellular membrane conductance as two capacitors in series, one capacitor for the basal membrane and the other for the apical membrane (Eq. 49) in which (J) is used to represent the imaginary number that results when taking the square root of a negative one and (f) represents the current frequency.

$${Z}_{m} = \frac{- j}{2 π f ( \frac{{C}_{m}}{2} )} \qquad ( 49 )$$

In practice, a membrane resistive component (Rₘ) due to ion transport should be present. As such, the membrane impedance needs to be modified. In one embodiment, the membrane impedance, Zₘ is formulated here as a capacitor and resistor in parallel for each membrane encountered vertically through the cell as follows.

$${Z}_{m} = \frac{1}{( \frac{1}{{R}_{m}} + j 2 π f {C}_{m} )} \qquad ( 50 )$$

6. Description of Levenberg-Marquardt Non-Linear Simulation (LM-NLS) Procedure

The LM-NLS may provide optimal estimates for the parameter solutions using the mathematical model in full complex form, magnitude form, a real valued form, or an imaginary form. The magnitude form creates and curve fits a single quantity by taking the square root of the sum of the squares of the real and imaginary parts respectively. The complex form curve fits two independent vector quantities of impedance in a balanced fashion by treating the real and imaginary parts separately. The full two dimensional complex parameter estimation process attempts to find optimum parameter values that minimize the error that exists between the simulated and experimental total cell covered responses in both the real and imaginary component simultaneously. The magnitude formulation of the parameter estimation process utilizes a one-dimensional real valued result that combines the real and imaginary components simultaneously while balancing the minimized error in both the real and imaginary components between the simulated and experimental responses. The real or imaginary optimization modes are one-dimensional as well, may optimize against the real or imaginary component when selected.

7. Error Evaluation

The real and imaginary experimental data of the cell-covered electrode (Z_{c}) and naked electrode (Zₙ) were measured at 23 frequencies between 25 to 60,000 Hz. The values of Z_{c} used in the model were measured 24 hrs after cell attachment at time points at which the endothelium achieved a steady state transendothelial resistance (TER). Values of Zₙ were measured after trypsinization of the cultured monolayers and replacement with fresh medium. A final calculated real and imaginary valued solution (Zₛ), using Eq. 51, was generated. The solutions generated are based on a set of parameters (parms), specifically α, R_{b} and Cₘ over the desired frequency range (f). The function to be fit with an optimum parameter set is of course Eq. 48, which describes the simulated or calculated impedance, Zₛ.
Zₛ=Zₛ(f, parms) (51)
The difference between the calculated and experimental cell impedance (Zₑᵣᵣₒᵣ) was defined as follows:

$${Z}_{error} = \frac{{Z}_{c}}{{Z}_{s}} - 1 \qquad ( 52 )$$

The error, Zₑᵣᵣₒᵣ, was used to plot a graphical representation of remaining error after a parameter fit was performed. A Chi² value was also calculated as a result of the analysis to define modeling error between the simulated and experimental data. A raw Chi² value (X²) was reported along with a reduced Chi² that divides the raw Chi² value by the number of data points involved (N) minus the degrees of freedom (DF) or the number of free parameters being fit. In the case of complex data the analysis additionally takes the square root of the final reduced Chi² value. The reduced Chi² value reported after an analysis takes the form as follows:

$${X}^{2} ( parm ) = {\sum }_{f = 1}^{n} {[ \frac{{Z}_{{s}_{f}} - {Z}_{{c}_{f}} ( parm )}{{σ}_{f}} ]}^{2} \cdot \frac{1}{N - DF} \qquad ( 53 )$$

Z_{c} represents the actual collected data and Zₛ represents the expected values for the observed values. The value assigned to σ_{f} is assumed to be one, a unit variance, because the measurement error in this situation is not known.

8. Simulation Procedure

A LabView based graphical user interface (GUI) provides user control over the simulation and parameter estimation routines embedded in the C++ dynamic link library. The GUI also provides numerical and graphical feedback of simulation and analysis results. The user supplies both cell covered and naked electrode data for graphical inspection along with an overlay of the simulated cell covered electrode. The user observes the results as the parameters are adjusted until a reasonably close match between the simulated and actual cell covered response occurs. This constitutes an initial guess for the parameter estimation process and can be used to update the simulation for greater accuracy.

9. Results

(a) A Comparison of Model Solution Parameters Derived by Visual Inspection and Levenberg-Marquardt Non-Linear Optimization

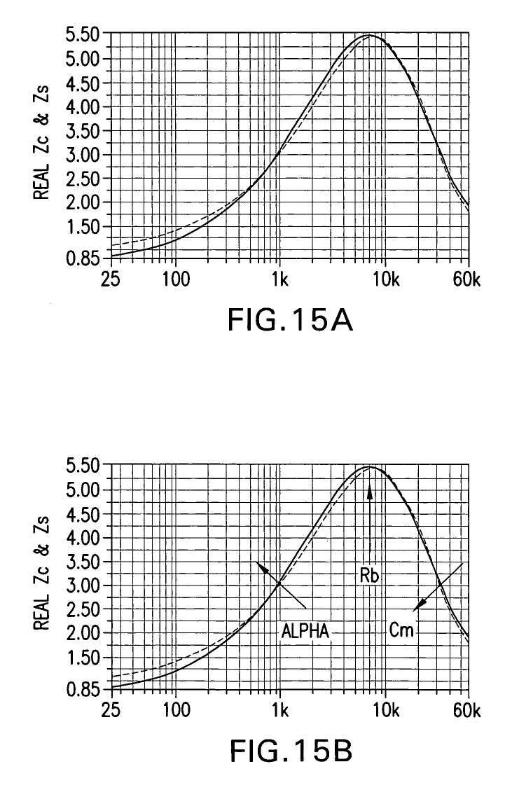

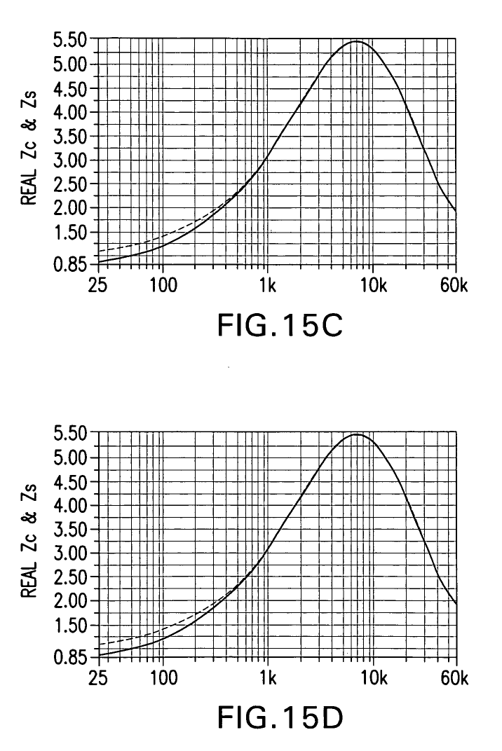

Model precision is dependent on the approach by which the numerical model is optimized to fit the experimental data. One approach is to find the solution parameters of the numerical model involves finding the best curve fit or match to the experimental measurement by using a visual inspection. FIGS. 15A-15D depict curve fitting between the calculated and experimental data between frequencies of 25 Hz to 60 kHz based on different optimized approaches. FIG. 15A depicts the fit between the calculated and experimental data when optimized by a typical visual inspection expressed as the ratio of the cell covered to the naked electrode resistance, where the ratio was greatest at approximately 7 kHz. The fit between calculated and experimental real data was best achieved at frequencies around the peak than at lower frequencies. FIG. 15B shows the heuristic directions the modeled response graph will change its shape when the model parameters for alpha (α), R_{b}, or Cₘ are increased in value respectively during an expert visual inspection. FIG. 15C shows the fit performed by an expert using the previously described rules of FIG. 15B. FIG. 15D shows the optimal fit performed by a numerical curve fitting routine provided by the LM-NLS.

As shown, a more accurate visually guided approach to derive cell membrane properties in the endothelium by systematic visual inspection requires consideration of a deterministic set of heuristics by which each solution parameter contributes to the calculated measurement of the real and imaginary data in a defined manner. As shown in FIG. 15B, α, R_{b} and Cₘ uniquely contribute to the real data in a defined fashion. If α is increased in value, a characteristic leftward and upward shift in the peak in real data occurs. Conversely, if α decreases in value, the calculated peak real data shifts rightward and downward. If the value of R_{b} increases, the net effect is to increase the peak of the real data in a vertical direction. Conversely, if R_{b} decreases, the net effect is to decrease the peak of the real data in a vertical direction. If the value of Cₘ increases, the effect shifts the right side of the peak in a downward direction. If a systematic approach that matches the data based on visual inspection is used to derive the calculated cell membrane properties, the resulting model fit (FIG. 15C) is similar in quality to a numerical fit obtained by an automated LM-NLS procedure (FIG. 15D).

Curve fitting between the calculated and the experimental real data appears similar by visual inspection between the different optimization approaches used. However, the derived values of α, R_{b} and Cₘ for the different visually based optimization approaches when compared to the LM-NLS procedure are distinctly different. For example, Table 4 compares the parameter estimates derived by a non-expert visual inspection (“laymen”) as shown in FIG. 15A and the parameter estimates derived by an expert visual inspection, FIG. 15C, with those derived by a Levenberg-Marquardt non-linear optimization approach, FIG. 15D. The right side of the table shows the ratio of the parameter values derived by the two visual inspection approaches when compared with the values derived by the LM-NLS approach. The values of α, R_{b} and Cₘ were similar between those derived by a LM-NLS procedure and a systematic visual inspection approach that considered the heuristic features of the real data, which would be considered the visual approach of an expert user. The percent accuracy defined as the percent ratio of the solution parameters derived by a heuristic approach to the values derived by the LM-NLS procedure was small (less than 5 percent). However, the values derived by a typical non-heuristic approach, which would be considered the visual approach of a laymen or inexperienced user, resulted in a much greater difference, which ranged from 8% for Cₘ; 32% for R_{b}; and 47% for a when compared with the LM-NLS procedure solutions. Taken together, these results demonstrate that deriving parameter solutions based solely on visual inspection lacks accuracy, and supports the requirement for numerical non-linear optimization approaches to derive parameter solutions of cell membrane properties.

TABLE 4
Comparison of α, R_{b} and Cₘ between derived
approaches and LM-NLS procedure

| | Parameter | | | Ratio of | % of Error in | | |
|---|---|---|---|---|---|---|---|
| Method | Estimates | | | Parameter/ | Parameter Estimate | | |
| Type | α | R_{b} | Cₘ | LM | α | R_{b} | Cₘ |
| Laymen | 1.96 | 3.63 | 3.46 | Expert/LM | 5.16 | −4.40 | −2.19 |
| Expert | 3.85 | 2.63 | 3.13 | Layman/LM | −46.46 | 31.95 | 8.12 |
| (LM) | 3.66 | 2.75 | 3.20 | | | | |

(b) The Effect of Frequency on Model Error and Solution Parameter Stability when Modeling the Experimental Real Data

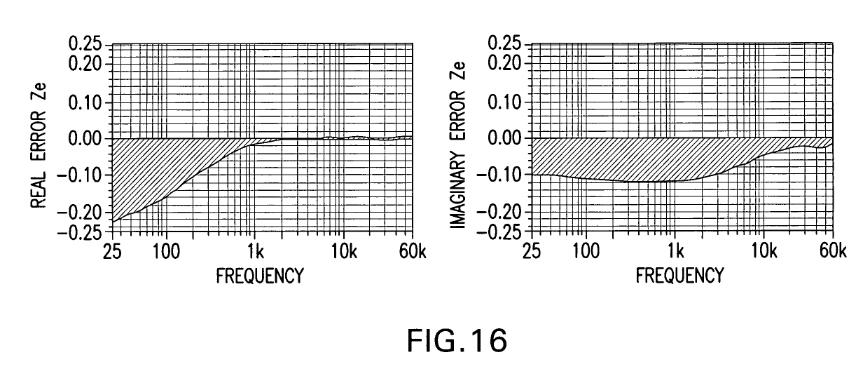

Since the model showed differences in accuracy in curve fitting the calculated real data to the experimental data as a function of frequency, the next step was to quantify this difference or error as a function of frequency. FIG. 16 depicts the error (Eq. 52) between the experimental real and imaginary data and the simulated real and imaginary data whose parameters were derived by the LM-NLS procedure. The left plot displays the Zₑᵣᵣₒᵣ of the real data and the right plot displays the Zₑᵣᵣₒᵣ plot of the imaginary data. As shown, Zₑᵣᵣₒᵣ is very small (less than 5 percent) for the real data except at frequencies below 2 kHz. In contrast, the range for relatively small error is a narrower bandwidth between 20 kHz to 60 kHz for the imaginary data. Taken together, these data suggest that deriving numerical solution parameters of cell membrane properties are affected by the frequency spectrum that is selected for the model. Also, the data suggests that the optimal frequency spectrum that is optimized to solve the model solution parameters is dependent on the type of data that is modeled.

Since the different levels of modeling error was observed as a function of frequency, model solution parameters were correlated with a measured normalized Chi² assessment at different frequency spectrums based on the real data. For this analysis, an upper frequency limit of 60 kHz was fixed, while the lower frequency marker was adjusted in predefined steps from 25 Hz to 30 kHz. Increasing the lower limit created a progressively smaller data set over a narrow frequency bandwidth that is sent to LM-NLS procedure to solve the membrane solution parameters. As shown in FIG. 17, estimations of real data solution parameters and Chi² values based on modeling a progressively smaller data subset between 25 Hz and 60 kHz are shown. The lower frequency included is plotted along the x-axis. The resulting parameter estimates are plotted along the left y-axis and the Chi² value is plotted along the right y-axis. The solution parameters and Chi² values were obtained by fixing the upper frequency at 60 kHz while the lower frequency was adjusted progressively upward towards higher frequencies. Chi², when modeling real data was greatest when using the entire frequency spectrum as anticipated. Yet, at a lower frequency marker between 2 to 10 kHz, the reduced Chi² achieved a relatively low plateau level.

The solution parameters for cell membrane properties based on real data were unstable when using a lower frequency marker below frequencies of 2 kHz, which corresponded to the highest levels of reduced Chi² error. Membrane property solutions were particular unstable for alpha. However, model solutions for cell membrane properties were quite stable over a frequency bandwidth in which the lower frequency ranged between 2 to 10 kHz, which corresponds to the frequency impedance spectrum that includes the left side, the peak, and the right side of the real data curve as shown in FIG. 15B. By increasing the lower frequency limit above 10 kHz, the reduced Chi² value decreased but was associated with less stable solutions of alpha. This observation was anticipated since real data at frequencies on the left side of the real data peak is required to resolve measurements of alpha as depicted in FIG. 15B. Taken together, this data indicates that there is a unique frequency spectrum that yields stable solution parameters when modeling the real data, and the solution parameters are unstable or unreliable when the model attempts to solve for solution parameters using a data set selected from a frequency spectrum using a lower frequency marker outside of 2-10 kHz.

(c) Frequency-Dependent Error and Solution Parameter Stability when Modeling the Experimental Imaginary, Magnitude, and Complex Data

If the numerical model is sufficient to derive model solution parameters, then the LM-NLS should produce the same solution parameters regardless of whether the LMS is modeling the real data, imaginary data, magnitude data or the complex data of the impedance. Before determining whether the LM-NLS can achieve stable solutions across these different data, the modeling stability affected by the frequency spectrum for each type of data is evaluated.

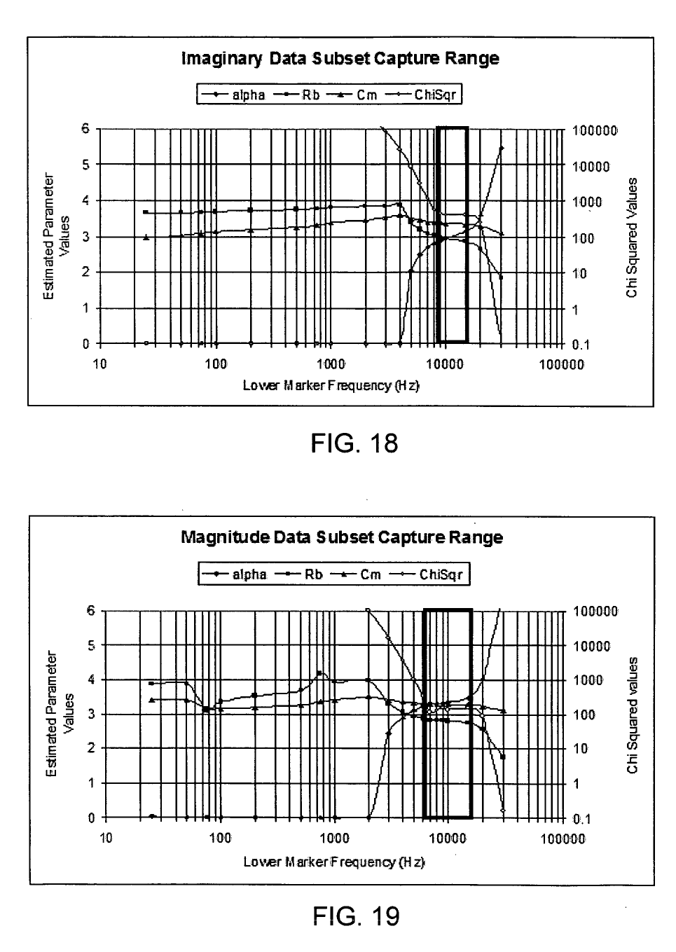

Referring to FIG. 18, the correlation between numerical solution parameters and reduced Chi² for imaginary data is shown. The estimations of imaginary data solution parameters and Chi² values based on modeling a progressively smaller data subset between 25 Hz and 60 kHz. The lower frequency included is plotted along the x-axis. The resulting parameter estimates are plotted along the left y-axis and the Chi² value is plotted along the right y-axis. The solution parameters and Chi² values were obtained by fixing the upper frequency at 60 kHz while the lower frequency was adjusted progressively upward towards higher frequencies. Modeling only the imaginary data demonstrate similarities and unique behaviors compared to modeling only the real data. Like modeling the real data, model stability and error was worse at low frequencies. However, the impedance spectrum that achieved the most stable model solutions at the least Chi² error was narrower than that observed when modeling the real data. Additionally, the frequency spectrum for identifying the most stable solution parameter at a low plateau Chi² error for the imaginary data (10 to 15 kHz) did not overlap with the impedance spectrum for real data (2 kHz to 10 kHz). Again, like the real data, modeling the imaginary data above 15 kHz resulted in a lower reduced Chi² error as well as a less stable solution for alpha.

In contrast, the best stability of the numerical model at the lowest plateau of reduced Chi² error based on the magnitude data overlapped in the frequency spectrum between the real and the imaginary data, as shown in FIG. 19. FIG. 19 depicts estimations of magnitude data solution parameters and Chi² values based on modeling a progressively smaller data subset between 25 Hz and 60 kHz. The lower frequency included is plotted along the x-axis. The resulting parameter estimates are plotted along the left y-axis and the Chi² value is plotted along the right y-axis. The solution parameters and Chi² values were obtained by fixing the upper frequency at 60 kHz while the lower frequency was adjusted progressively upward towards higher frequencies. The most stable numerical solution parameters based on the magnitude data was observed between 7 kHz to 15 kHz.

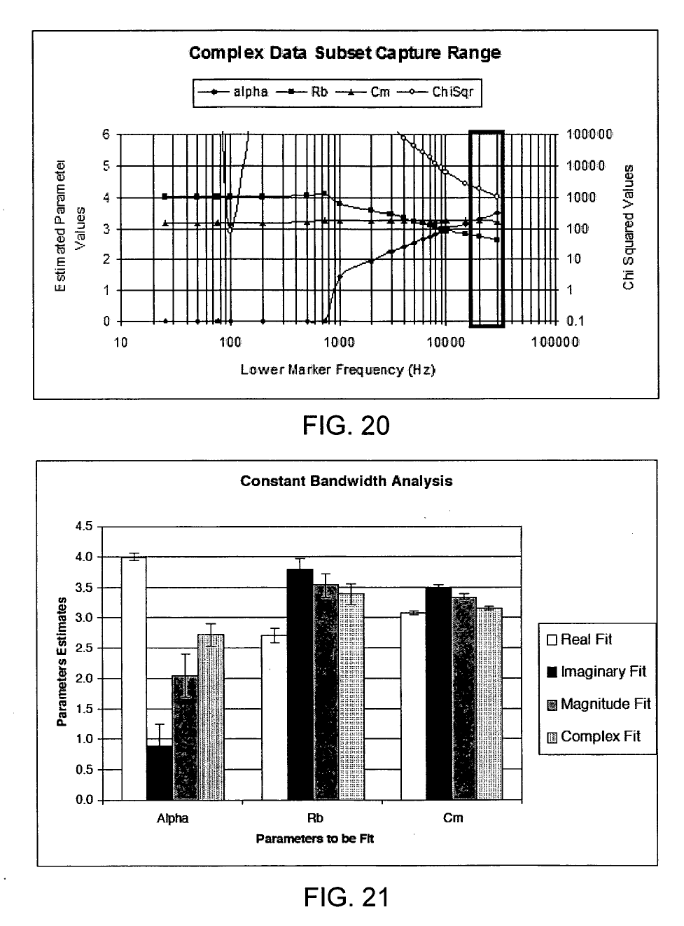

The best stability of the numerical model at the smallest reduced Chi² error based on the two dimensional complex formulation including both real and imaginary data simultaneously, was observed at a narrow frequency bandwidth between 20 kHz to 30 kHz, as shown in FIG. 20. FIG. 20 depicts estimations of complex data solution parameters and Chi² values based on modeling a progressively smaller data subset between 25 Hz and 60 kHz. The lower frequency included is plotted along the x-axis. The resulting parameter estimates are plotted along the left y-axis and the Chi² value is plotted along the right y-axis. The solution parameters and Chi² values were obtained by fixing the upper frequency at 60 kHz while the lower frequency was adjusted progressively upward towards higher frequencies. The lower frequency limit that yielded the most stable solutions is highlighted with the black box and is between 15 kHz to 30 kHz. Taken together, these data demonstrate that modeling stability is dependent on the frequency spectrum, and the targeted frequency spectrum data that the LM-NLS optimizes is dependent on the type of data that is modeled.

(d) A Comparison of Constant Bandwidth Versus Minimum Chi² Analysis with Consideration for Model Formulations of Real, Imaginary, Magnitude, and Complex

To illustrate how modeling stability is a function of the type of data that is modeled and the frequency spectrum used, the mean solution parameters derived by the LM-NLS for the real, imaginary, magnitude and complex data based on selecting the same frequency bandwidth that is optimal for the real data (2 kHz to 60 kHz) was compared, as shown in FIG. 21. FIG. 21 compares the mean (±SE) solution parameters based on real, magnitude, imaginary, and complex data of the cell covered and the naked electrode obtained by exposing the cells to trypsin. The model solution parameters were obtained by selecting a fixed frequency bandwidth from 2 kHz to 60 kHz for all types of data. The sample size was N=14. As anticipated, selecting the same bandwidth to model all types of impedance data resulted in very dissimilar solution parameters. The LM-NLS had the most difficulty in identifying a reproducible solution for the alpha parameter, which is most sensitive to frequency. The model solutions for R_{b} and Cₘ identified by the LM-NLS procedure were slightly higher for the imaginary, magnitude and complex data than for the real data.

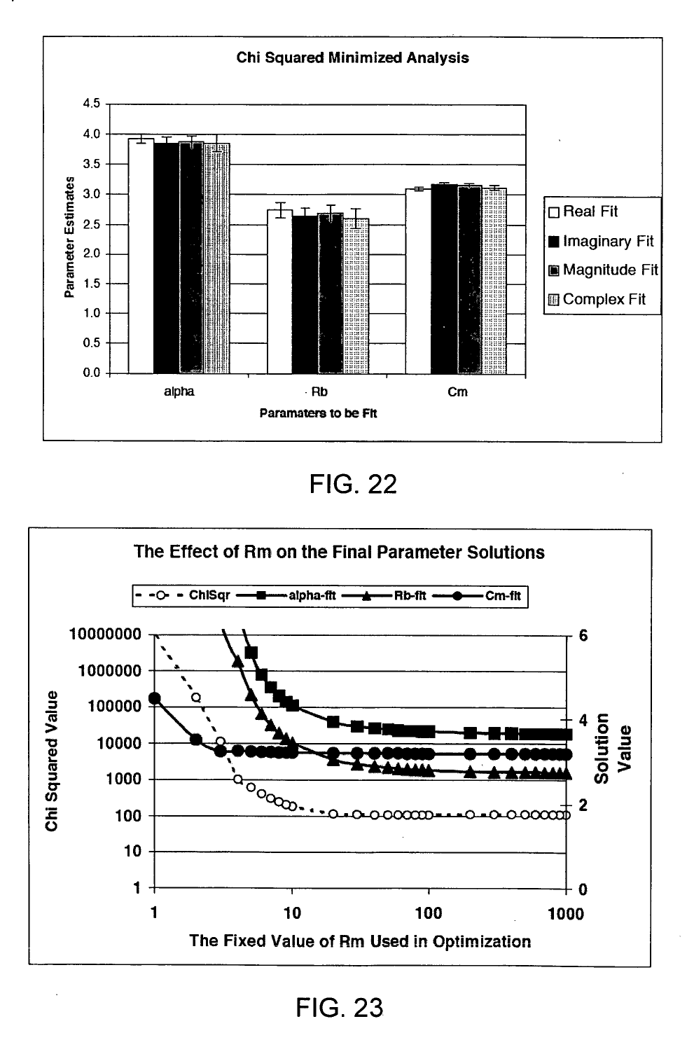

In contrast, the model achieved the most stable solution parameters when the LM-NLS procedure identified the parameters based on the criteria of choosing the frequency bandwidth that provided a low reduced Chi² error, as shown in FIG. 22. The model solution parameters were obtained by selecting a variable frequency bandwidth chosen based on a criterion that minimized the Chi² values in each analysis. The sample size was N=14. Under these conditions the mean solution parameters are more reproducible, which demonstrate model stability. Using this approach, the model identified very reproducible solution parameters between modeling the real, imaginary, magnitude, and complex data. Taken together, this data demonstrates that the model can solve for stable solution parameters irrespective of the type of impedance data formulation that is used based on an approach that selects the frequency bandwidth that minimizes error.

(e) The Effect of Including Fixed Values of Transmembrane Resistance Rₘ on Parameter Solution Stability

In the Giaever report, transcellular membrane conductance can be characterized as two capacitors in series, one capacitor for the basal membrane and the other for the apical membrane (Eq. 49). A practical modification includes a membrane resistive component due to ion transport (Eq. 50). Including this parameter in the list of parameters to be fit generally resulted in severe instabilities in resulting parameter estimates. Fixing it to a certain value restored robustness to the solution estimation process. If values of transcellular membrane conductance (Rₘ) at various values (FIG. 23) were fixed, the effect on perturbing the results of parameter estimation on the remaining parameters is seen. For fixed values of Rₘ greater than 200 Ohms/cm², the resulting parameter estimates for the remaining parameters is less than 1% of difference (0.03% for Cₘ; 0.83% for R_{b}; and 0.57% for α) when comparing to the asymptotic values to the right side of the FIG. 23. This data shows that if the likely value for transcellular membrane conductance (Rₘ) is fixed to a value greater than 200 Ohms/cm² then the resulting analysis parameter solutions and Chi² error will not be affected.

10. Discussion

The data shows that the estimations of model solution parameters of cell membrane properties are dependant on the frequency spectrum and the type of impedance data submitted to the LM-NLS. Modeling stability was assessed by examining how the LM-NLS estimated cell-membrane parameters and Chi² error for the real, imaginary, complex and magnitude transendothelial impedance data as a function of frequency bandwidth. For each type of experimental data, model solution parameters were dependent on unique frequency spectrums. The frequency spectrums that achieved the most optimal solutions at low plateau level Chi² error were non-overlapping when the LM-NLS estimated solution parameters from the real and imaginary experimental data. Optimization of solution parameters based on the magnitude and complex modes took on the partial character of the real and imaginary formulations. The frequency bandwidths to identify stable solution parameters based on magnitude and the complex data did not represent the sum of the bandwidths of the real and imaginary data alone. Since the optimal frequency bandwidths for the real and imaginary were non-overlapping, the real and the imaginary data have different impact on the magnitude and the complex data. The magnitude data represents a one-dimensional quantity formed out of the real and imaginary components. If the bandwidths for identifying stable solution parameters for the real and imaginary components do not overlap, then it would be anticipated that the bandwidth for identifying stable solutions based on magnitude data should not increase, which was the case. In contrast, the complex mode formulation represents a two-dimensional quantity of the real and imaginary input components, which requires satisfying the model for both data simultaneously. Since the frequency bandwidths for the real and imaginary data do not overlap, it is expected that optimization algorithms to derive stable solutions at minimal error would occur over a narrow frequency bandwidth, which was the case.

By choosing the appropriate bandwidths for the analysis, and by minimizing the Chi² result in each case, the LM-NLS achieved very consistent results. When analyzing the model, the data shows that the extrapolation error also needs to be minimized. This notion is supported by the notion that the cell membrane parameters were consistent between the real, imaginary, complex, and magnitude data sets when a strategy was used to identify the frequency spectrum that minimized error in terms of the measured Zₑᵣᵣₒᵣ or Chi². Graphical plots using the measured Zₑᵣᵣₒᵣ extracted the actual extrapolation error. In contrast, when a fixed frequency spectrum that was suited for the real data was applied to the imaginary, magnitude and complex data sets, there was added variability in the estimates in the cell membrane parameters, which indicates such an approach leads to greater modeling error and parameter estimate instability. In particular, the greatest variability in the solution estimates occurred in cell-matrix adhesion when modeling the different types of impedance data. Further, the model solution parameter instability was most frequently observed for the cell-matrix adhesion parameter in a frequency-dependent fashion. As such, extremely low values for Chi² can be achieved if not enough data are used for fitting. For example, if only three data points were used to derive solution parameters, a Chi² of zero would likely result. However, there is a tradeoff of choosing too small of a data subset to minimize Chi², which would unduly affect the model predictions for the larger original data set.

Under ideal conditions the instrumental noise would also be known at each frequency and the model would provide a true representation of the experimental system. In these cases, the successful optimization of the model parameters would produce a reduced Chi² on the order of unity. Frequency data points with large deviations would be weighted less than those with smaller deviations. In cases where the instrumental noise is not known, it is assumed that noise remains constant. If the underlying noise distribution is frequency dependent, large numerical instabilities can arise during the optimization process and determining a stable range of sampling frequencies would be necessary. Frequency dependent systematic errors can introduce an additional complication.

Although introducing noise measurements into the Chi² analysis can improve the stability, the noise measurements can introduce additional numerical artifacts. In cases where the noise fluctuations are insignificant, singularities could arise during the computation. This can occur, for example, when filtering successfully reduces the electrical fluctuations to the level of the analog to digital discretization level. If the noise is non-Gaussian, the estimation would not be maximum likelihood. Filtering, sixty-Hertz noise, and other artifacts, for example, could introduce non-Gaussian noise into the data.

By their nature, nonlinear optimization algorithms can produce optimized parameters that are dependent on the starting parameters. By preceding the nonlinear optimization with a visual fit a more appropriate starting parameter can be chosen.

Identifying the optimal frequency bandwidth was first accomplished by identifying the upper and lower frequency band at which Zₑᵣᵣₒᵣ was typically low (below 10 percent) by first applying the LM-NLS procedure to the entire experimental data set between 25-60,000 Hz. Next, the optimization algorithms were repeated with the restricted data subset at the targeted frequency bandwidth. The data demonstrates that using criteria that derived cell-membrane parameters based on minimizing the Chi² error in the LM-NLS optimization resulted in the most stable and reproducible cell-membrane parameters.

The data also illustrate the principle that deriving solution parameters based on visual inspection criteria alone is prone to potential error. Choosing a visual fit between the calculated model and the experimental data without regard to the heuristic guided approach potentially leads to significant error. Yet, even with a heuristically guided approach, some remaining error cannot be eliminated. The current data shows a numerically guided approach that automates and finds model solution parameters based on recognized numerical optimization approaches.

The impact of the membrane resistive component of the cell monolayer impedance was also evaluated. As defined in Eq. 50, transcellular membrane impedance is inversely related to the value of Rₘ. The impact of Rₘ on model solution parameters of α, R_{b} and Cₘ as well as Chi² becomes inconsequential as values of Rₘ exceeds 200 Ω/cm². The limited role of Rₘ is consistent with the empiric experience of why it is possible to measure the very low ion conductance by patch clamping. In order to measure the typical pA levels of ion channel conductance based on Ohms law, Rₘ must be large so that it can be treated as a constant.

To compensate for any systematic error and achieve modeling stability, the present disclosure provides computational algorithms that can select a subset of the original database at variable frequency bandwidths. In this fashion, data in the frequency range below 2 kHz where the error is most prevalent may be excluded.

There are several potential explanations for the systematic error between the calculated and the experimental data, which requires understanding the assumptions of the experimental measurement and the numerical model proposed by Giaever. First, there may be systematic error introduced by the instrumentation circuit, which affects how biological activity is measured.

Second, the model assumes that there is no drift in the instrumentation system. Zₙ is not modeled but is simply mathematically divided into Z_{c}, which only holds true if both Zₙ and the transendothelial impedance are both resistor and capacitors in series. If either the monolayer or electrode does not behave as resistor and capacitor in series, then different numerical expressions for Zₛ and Z_{c} are required. Further, the model must assume that Zₙ behaves as a constant and exhibits no measurable drift over time. If there is significant electrical drift, then Zₙ needs to be numerically modeled. Thus, for this reason, the inventors have chosen experimental data for Zₙ after removing cells off the electrode with trypsin in order to reduce Zₑᵣᵣₒᵣ.

Third, the model assumes that the cell geometry is disk shape in which there are gaps between cells. Since endothelial cells are not disk shape, it remains to be validated whether the model solution parameters and model stability is affected by selecting a different cell geometry.

Fourth, the LM-NLS optimization assumes that there is a Gaussian distribution of noise across all measured frequencies. For these analyses, the a value for the Chi² was assumed to be unity since α was unknown and was not experimentally measured. If there is variable distribution of noise as a function of frequency, then the optimization algorithms require a weighted function to compensate for frequency-dependent noise levels.

The results of the data of Example 2 show that modeling transendothelial impedance as a circuit that consists of a repeating pattern of disks and a resistor-capacitor in series is not sufficient in modeling the entire impedance frequency spectrum. The present data indicate that a more complicated numerical model is required to characterize the entire impedance spectrum between 25-60,000 Hz. More complicated models should provide a more complete fit between the simulated and the experimental measurement at all measured frequencies and for both the real and imaginary data.

The experimental measurement derived by ECIS does not directly provide indices of cell-cell adhesion, cell-matrix adhesion and membrane capacitance. Rather these parameters must be derived by numerical modeling because the electrode area is greater than the diameter of a single cell. Changes in transendothelial resistance in response to physiological stimuli is frequently assumed that such changes are targeted at cell-cell adhesion sites. However, it is noted that PKC activation initiates a disruption in barrier function that primarily target cell-matrix adhesion sites. Additionally, it is noted that heterologous expression of low-molecular weight caldesmon attenuated adenoviral-mediated reduction in transcellular resistance in cultured fibroblasts predominately through effects on cell membrane capacitance. Only by numerical modeling the experimental transcellular impedance at multiple frequencies can one elucidate and localize the membrane sites by which inflammatory stimuli and pathogens mediate cellular injury.

The numerical model and algorithms presented in this disclosure may be utilized for several essential applications for cell biologists and cell physiologists. First, the model can be applied to evaluate how exogenous physiological and pathological stimuli regulate endothelial and epithelial barrier function. Also, the numerical model can be utilized to quantify and evaluate how cell-cell and cell-matrix adhesion contribute to cell motility and wound repair under different experimental conditions in cultured cell systems. Second, the model can be applied to quantify and elucidate with precision cell-cell interactions between leukocyte-endothelial and pathogen-host interactions for example). Third, the model may be useful to elucidate signal transduction pathways that regulate cell membrane properties under different experimental conditions. Fourth, the model may have utility to precisely evaluate how genomics and proteonomics of the cytoskeleton regulate cell-membrane properties in intact living cells in which the behavior of the cytoskeleton may not be adequately predicted from in vitro bioinformatics tools. Fifth, the numerical model can be used to evaluate molecular mechanisms of drug toxicity. The numerical model could be multiplexed with electrical and optical-based assays to evaluate molecular mechanisms of drug therapeutics and toxicity. Ultimately, the accuracy of these analyses and simulations is dependent on reliable numerical models and computational algorithms that not only resolve model solution parameters but also assess modeling stability and error for those model solutions. Without assessments of modeling stability and error, the experimentalist cannot derive confidence in the model solution parameters and make appropriate interpretations of cell membrane properties.

In summary, a comprehensive assessment of modeling error and stability based on a numerical model that characterizes transcellular impedance across a cell-covered electrode and as a resistor and capacitor in series. The inventors have demonstrate that there are potential data type and frequency-dependent modeling instabilities and systematic errors in the solution parameters. By understanding these experimental factors, reproducible and reliable numerical solutions of cell-cell and cell-matrix adhesion and membrane capacitance can be derived from the measured transendothelial impedance. Employing a numerically stable parameter estimation process and including the appropriate range of frequencies in the parameter estimation process can obtain more accurate and reproducible parameter estimates of cell membrane properties. Since the diameter of the electrode exceeds the diameter of a single cell, the experimental measurement of ECIS cannot derive spatial measurements of cell-membrane properties without reliable numerical models and computational algorithms. By quantifying the sources of error and parameter estimate instabilities, a method of impedance spectroscopy for determining in vitro cell monolayer properties can more precisely elucidate the cytoskeletal-membrane properties that regulate endothelial barrier function.

With the benefit of the present disclosure, those having skill in the art will comprehend that techniques claimed herein may be modified and applied to a number of additional, different applications, achieving the same or a similar result. The claims that follow cover all such modifications that fall within the scope and spirit of this disclosure.

### REFERENCES

Each of the following references is hereby incorporated by reference in its entirety:
Alexander et al. J. Cellular Physiology, 156:610-618, 1993.
Baldursson et al., Amer. J. Physiol. Lung Cell. Molec. Physiology, 279:L835-841, 2000.
Bates and Watts, In: Nonlinear regression analysis and its applications, N.Y., Wiley, xiv:365, 1988.
Bums et al., J. Cell Science, 113:45-57, 2000.
Carbajal et al., Amer. J. Physiol.—Cell Physiology, 279:C195-204, 2000.
Carbajal and Schaeffer, Jr., Amer. J. Physiol., 277:C955-964, 1999.
Carson et al., Amer. J. Physiol., 257:L259-264, 1989.
Ellis et al., Amer. J. Physiol., 276:C38-C45, 1999.
Gainor et al., Amer. J. Physiol. Heart Circ. Physiol., 281:H1992-H2001, 2001.
Geroski and Hadley, Current Eye Research, 11:61-72, 1992.
Giaever and Keese, Proc. Natl. Acad. Sci. USA, 90:1634, 1993.
Giaeve and Keese, Physica D, 38:128-133, 1989.
Giaever and Keese, Proc. Natl. Acad. Sci. USA, 88:7896-7900, 1991.
Giaever and Keese, Proc. Natl. Acad Sci. USA, 81:3761-3764, 1984.
Giaever and Keese, IEEE Trans. Biomed. Eng. BME, 33:242-247, 1986.
Haxhinasto et al., Amer. J Physiol.—Cell Physiol., 287:C1125-C1138, 2004.
Iyer et al., Amer. J. Physiol. Lung Cell. Molec. Physiology, 286:L1143-1153, 2004.
Johnson, J. Applied Physiol., 65:2302-2312, 1988.
Johnson et al., Amer. J. Physiol., 256:H1012-1022, 1989.
Johnson and Wichem, In: Applied multivariate statistical analysis, New Jersey: Prentice-Hall Inc., 594, 1982.
Kataoka et al., Proc. Natl. Acad. Sci. USA, 99:15638-43, 2002.
Keese and Giaever, IEEE Eng. Med. Bio., 402-408, 1994.
Langeler and VanHinsbergh, Amer. J. Physiol., 260:C1052-C1059, 1991.
Lo et al., Experi. Cell Res., 250:576-580, 1999.
Lo et al., Biophys. J., 69:2800-2807, 1995.
Lo et al., Experi. Cell Res., 204:102-109, 1993.
Lum et al., Am. J. Physiol. Cell Physiol., 282:C59-C66,2002.
Moyetal., J. Clin. Invest., 97:1020-1027, 1996.
Moy et al., Am. J. Physiol. Lung. Cell Mol. Physiol., 278:L888-L898, 2000.
Moy et al., Amer. J. Physiol.—Heart Circul. Physiol., 292:H21-H29, 2002.
Moy et al., Amer. J. Physiol.—Lung Molec. Physiol., 297:L153-167, 2004.
Noiri et al., Am. J. Physiol., 274:C236-C244,1998.
Noria et al., Circulation Res., 95:504-514, 1999.
Patterson et al., Amer. Rev. Respir. Disease, 140:1052-1062, 1989.
Patterson et al., Microvascular Res., 48:212-235, 1994.
Phelps and DePaola, Am. J. Physiol. Heart Circ. Physiol., 278:H469-H476, 2000.
Press et al., In: Numerical recipes in c the art ofscientific computing, Cambridge: Cambridge University Press, 994, 1999.
Sandoval et al., J. Physiology, 533:433-445, 2001.
Shasby et al., Amer. Rev. Respir. Disease, 127:72-76, 1983.
Shi etal., Endothelium, 6:153-171, 1998.
Tiruppathi et al., Proc. Natl. Acad. Sci. USA, 89:7919-7923,1992.
Tschugguel et al., Pflugers Arch—Eur. J. Physiol., 430:145-147, 1995.
Vouret-Craviari et al., Molec. Biol. Cell, 9:2539-2653, 1998.
Wegener et al., Experi. Cell Res., 259:158-166, 2000.
Wegener et al., Pflugers Arch—Eur. J. Physiol., 437:925-934, 1999.
Wetering et al., Am. J. Physiol. Cell Physiol., 285:C343-C352, 2003.
Wojciak-Stothard et al., J. Cell Science, 114:1343-1355, 2001.
Wojciak-Stothard et al., Vascular Pharmacology, 39: 197-199, 2002.
Xiao et al., Anal. Chem., 74:5748-5753, 2002a.
Xiao et al., Anal. Chem., 74:1333-1339, 2002b.
Yuan et al., Amer. J. Physiol., 272:H1437-1443, 1997.

1. A method comprising:
(a) determining a cell membrane property derived from a transcellular impedance measurement;
(b) determining a stability of the measurement; and
(c) determining an error of the measurement.

2. The method claim 1, where the cell membrane property comprises cell adhesion and where determining the cell adhesion comprises measuring a cell-cell adhesion.

3. The method of claim 2, where determining the cell adhesion comprises measuring a cell-matrix adhesion.

4. The method of claim 1, where the cell membrane property comprises cell membrane capacitance and cell membrane resistance.

5. The method of claim 1, where determining the stability comprises performing a statistical calculation.

6. The method of claim 1, where determining an error comprises a performing a Chi² calculation.

7. The method of claim 1, further comprising determining a precision of the measurement.

8. The method of claim 1, where the transcellular impedance comprises transcellular impedance of cultured cells.

9. The method of claim 8, where the cultured cells comprise cells grown on a microsensor.

10. The method of claim 9, where the microsensor comprises a microscopic biosensor.

11. The method of claim 9, where the microsensor comprises a microelectrode.

12. The method of claim 1, where (a) comprises utilizing a non-linear optimization algorithm.

13. The method of claim 12, where the non-linear optimization algorithm comprises a Levenberg-Maquardt non-linear optimization algorithm.

14. The method of claim 12, where the non-linear optimization algorithm comprises a least-squared non-linear optimization algorithm.

15. The method of claim 1, where (a) comprises utilizing real and imaginary data pertaining to the transcellular impedance.

16. The method of claim 1, where (a) comprises utilizing complex and magnitude data pertaining to the transcellular impedance.

17. The method of claim 1, where (a) comprises modeling a cell using a geometric shape, the geometric shape being selected from the group consisting of a disk, a square, a rectangle, a parallelogram, and an ellipsoid.

18. The method of claim 1, where (a) further comprises eliminating low frequency data pertaining to the transcellular impedance.

19. The method of claim 1, where (a) further comprises statistically weighting low frequency data pertaining to the transcellular impedance.

20. The method of claim 1, where (a) further comprises modeling an accumulating error in an electrode property over time.

21. A program storage device readable by a machine, tangibly embodying a program of instructions executable by the machine to perform the method steps of claim 1.

22. A method comprising:
determining a cell membrane property from a transcellular impedance measurement, where determining the cell membrane property comprises:
(a) modeling real and imaginary data pertaining to the transcellular impedance; and
(b) using a geometric shape to model the shape of the cell.

23. The method of claim 22, further comprising utilizing a Levenberg-Maquardt non-linear optimization algorithm.

24. The method of claim 22, further determining a stability of the transcellular impedance.

25. The method of claim 22, further determining an error of the transcellular impedance.

26. The method of claim 25, where determining an error comprises a performing a Chi² calculation.

27. The method of claim 22, where the geometric shape is selected from the group consisting of a disk, a square, a rectangle, a parallelogram, and a ellipsoids.

28. A method comprising:
(a) determining membrane capacitance and membrane resistance of a cell from a transcellular impedance measurement.

29. The method of claim 28, where (a) comprises utilizing a Levenberg-Maquardt non-linear optimization algorithm.

30. The method of claim 28, where (a) further comprises modeling the cell using a geometric shape, the geometric shape being selected from a group consisting of a disk, a square, a rectangle, a parallelogram, and a ellipsoids.
